(19) United States
(12) Patent Application Publication
(10) Pub. No.: US 2023/0149719 A1
(43) Pub. Date: May 18, 2023

(54) CARDIAC THERAPY CONFIGURATION USING EXTERNAL ELECTRODES

(71) Applicant: Medtronic, Inc., Minneapolis, MN (US)

(72) Inventors: Subham Ghosh, Blaine, MN (US); Elizabeth A. Schotzko, Blaine, MN (US)

(21) Appl. No.: 17/982,638

(22) Filed: Nov. 8, 2022

Related U.S. Application Data
(60) Provisional application No. 63/280,338, filed on Nov. 17, 2021.

(51) Int. Cl. A61N 1/368; A61N 1/372; A61N 1/365; A61B 5/33; A61B 5/282

(52) U.S. Cl. CPC A61N 1/3682; A61B 5/33; A61B 5/282; A61N 1/36585; A61N 1/37247

(57) ABSTRACT

Systems and methods are described herein for configuration of cardiac therapy. The systems and methods may select, or determine, a plurality of different configuration parameters based electrical activity monitored or measured using a plurality of external electrodes. For example, the systems and methods may select, or determine, a left ventricular pacing vector, adaptive or non-adaptive pacing therapy, an interventricular pacing delay, and a atrioventricular pacing delay.

### RELATED APPLICATIONS

This application claims the benefit of U.S. Provisional Application Serial Number 63/280,338, filed Nov. 17, 2021, the entire contents of each of which are incorporated herein by reference.

### TECHNICAL FIELD

The disclosure herein relates to systems and methods for use in configuration of cardiac therapy using external electrode apparatus.

### BACKGROUND

Implantable medical devices (IMDs), such as implantable pacemakers, cardioverters, defibrillators, or pacemaker-cardioverter-defibrillators, provide therapeutic electrical stimulation to the heart. IMDs may provide pacing to address bradycardia, or pacing or shocks in order to terminate tachyarrhythmia, such as tachycardia or fibrillation. In some cases, the medical device may sense intrinsic depolarizations of the heart, detect arrhythmia based on the intrinsic depolarizations (or absence thereof), and control delivery of electrical stimulation to the heart based on the intrinsic depolarizations.

IMDs may also provide cardiac resynchronization therapy (CRT), which is a form of pacing. CRT involves the delivery of pacing to the left ventricle, or both the left and right ventricles, as well as the atria. The timing and location of the delivery of pacing pulses to the ventricle(s) may be selected to improve the coordination and efficiency of ventricular contraction. Additionally, CRT may be delivered in adaptive or non-adaptive configurations.

Adaptive CRT may be generally described as CRT that adjusts in real time, “on the fly,” in response to one or more parameters monitored by an IMD. For instance, adaptive CRT may change the type of pacing therapy being delivered based on heart rate. For example, biventricular pacing therapy may be delivered when a patient’s heart rate is above 100 beats per minute and left ventricular only pacing therapy may be delivered when a patient’s heart rate is less than or equal to 100 beats per minute. It is to be understood that these are only a couple examples of adaptive CRT, and adaptive CRT may utilize measured, intrinsic atrioventricular delays and heart rate in conjunction with each other as well as other metrics to adjust CRT in real time. Conversely, non-adaptive CRT may utilize a fixed mode that is not adjustable in real time or “on the fly.” For example, non-adaptive CRT may be one of biventricular pacing therapy, left ventricular-only pacing therapy, and right ventricular-only pacing therapy, etc. and may utilize fixed atrioventricular and/or fixed interventricular pacing delays (e.g., a fixed time value, a fixed percentage of intrinsic atrioventricular delay, a fixed percentage of intrinsic interventricular delay, etc.).

Systems for implanting medical devices may include workstations or other equipment in addition to the implantable medical device itself. In some cases, these other pieces of equipment assist the physician or other technician with placing the intracardiac leads at particular locations on or in the heart. In some cases, the equipment provides information to the physician about the electrical activity of the heart and the location of the intracardiac lead.

Systems and devices, e.g., that perform CRT, may offer a lot of different pacing parameters or programming options, which include different pacing electrodes and vectors (e.g., quadripolar leads), different pacing configurations (e.g., biventricular, left ventricle only), different pacing timings (e.g., atrioventricular pacing delay, interventricular pacing delay for biventricular pacing), etc. External electrode apparatus can measure the efficacy of resynchronization for a given parameter set and may help tailor cardiac therapy device programming to maximize the degree of resynchronization. However, there are many permutations of pacing parameters, and hence, an efficient way to arrive at the optimal pacing parameter set in a given patient may be important for increasing the clinical efficiency.

### SUMMARY

The illustrative systems and methods described herein may be configured to assist a user (e.g., a physician) in evaluating and configuring cardiac therapy (e.g., cardiac therapy being performed on a patient during and/or after implantation of cardiac therapy apparatus). In one or more embodiments, the systems and methods may be described as being noninvasive. For example, in some embodiments, the systems and methods may not need, or include, implantable devices such as leads, probes, sensors, catheters, implantable electrodes, etc. to monitor, or acquire, a plurality of cardiac signals from tissue of the patient for use in evaluating and configuring the cardiac therapy being delivered to the patient. Instead, the systems and methods may use electrical measurements taken noninvasively using, e.g., a plurality of external electrodes attached to the skin of a patient about the patient’s torso.

The illustrative systems and methods may include the use of an external electrode apparatus, or ECG belt, that is applied to the torso of a patient to determine optimal synchrony during biventricular and left ventricular pacing. The illustrative systems and methods may be described as providing a robust and efficient optimal parameters selection, or determination, using an electrocardiogram (ECG) apparatus. It may be described that the illustrative systems and methods provide a sequence of steps to be implemented as a “workflow wizard” for cardiac therapy configuration. The workflow wizard can be executed automatically by a standalone device (such as, e.g., a cellular telephone, a tablet computer, etc.) or an integrated device where the device communicates (e.g., wirelessly using BLUETOOTH, WIFI, etc.) with the system (e.g., controller, amplifier, electrode apparatus, etc.) and can proceed to perform the illustrative processes described herein to, e.g., determine optical left ventricular pacing vector, pacing therapy type (e.g., adaptive CRT, non-adaptive CRT, etc.) interventricular (VV) pacing delay, and/or atrioventricular (AV) pacing delay. Further, the illustrative systems and methods provided herein may be described as providing a minimum number of tests to get to a patient-specific optimal CRT configuration based on data obtained, gathered, measured, and/or monitored, using the external electrode apparatus. In other words, this disclosure describes one or more protocols to be included in a software post-implant workflow wizard using external electrode apparatus that can help get to optimal pacing parameters (e.g., pacing vector, AV pacing delay, VV pacing delay, etc.) in under 15 tests.

One illustrative method may include measuring the patient’s intrinsic atrioventricular delay and generating an initial AV pacing delay from the pacing device. Then, the method may find the optimal vector pacing vector based on best electrical heterogeneity change by testing four left ventricular cathodes electrodes at the initial AV pacing delay and a VV pacing delay of 0. Then, adaptive pacing therapy may be delivered (e.g., left ventricle only pacing or biventricular pacing is performed based on patient’s intrinsic delay and heart rate. Next, adaptive pacing may be turned off and standard biventricular pacing therapy may be delivered. The optimal VV pacing delay may be determined or selected by delivering biventricular pacing delay using the initial AV pacing delay and six different VV pacing delays such as, e.g., -20 milliseconds (ms) (e.g., negative VV pacing delays pace the right ventricular before the left ventricle), 0 ms, 20 ms, 40 ms, 60 ms, and 80 ms. If patient’s intrinsic standard deviation of activation times (SDAT) is less than 24 ms, the VV pacing delay of -20 ms may be skipped or not performed. If the patient’s intrinsic average left ventricular or thoracic surrogate electrical activation times (LVAT) is less than 28 ms, the VV pacing delay of 80 ms may be skipped or not performed. The optimal VV pacing delay may be retained and three AV pacing delays may be tested. For example, biventricular pacing therapy maybe be delivered using an AV pacing delay at 40%, 60% and 80% of patient’s intrinsic AV delay. The AV pacing delays may be sorted based on SDAT improvements (e.g., from greatest reduction to least reduction) to arrive at the best AV pacing delay. As a result, each of the difference configurations performed may have been used to generate a SDAT, which may be used to select the optimal pacing configuration (e.g., left ventricular pacing vector, adaptive or non-adaptive pacing therapy, fixed AV pacing delay, and fixed VV pacing delay). Additionally, although this embodiment may evaluate adaptive or non-adaptive pacing therapy determination prior to AV pacing delay and VV pacing delay determination, it is to be understood that these processes may be performed in different orders. For example, VV pacing delay may be determined first, followed by AV delay, and then adaptive or non-adaptive pacing therapy.

One illustrative system, e.g., for use in cardiac therapy configuration, may include a computing apparatus. The computing apparatus may include processing circuitry and may be configured to obtain external electrical activity measured from tissue of a patient and generate electrical heterogeneity information (EHI) based on the obtained electrical activity. The computing apparatus may be further configured to select a left ventricular pacing vector of a plurality of different left ventricular pacing vectors based on the generated EHI from the electrical activity measured during delivery of pacing therapy using the plurality of different left ventricular pacing vectors. Each different left ventricular pacing vector may use a combination of pacing electrodes different from the remaining different left ventricular pacing vectors. The computing apparatus may be further configured to select one of adaptive pacing therapy or non-adaptive pacing therapy based on the generated EHI from the electrical activity measured during delivery of adaptive pacing therapy using the selected left ventricular pacing vector. The adaptive pacing therapy may adjust one or more pacing timing intervals based on one or both of intrinsic atrioventricular conduction and heart rate. In response to selection of adaptive pacing therapy, the computing apparatus may be further configured to provide an indication that the pacing therapy is complete. In response to selection of non-adaptive pacing therapy, the computing apparatus may be further configured to select one interventricular pacing delay of a plurality of different interventricular pacing delays based on the generated EHI from the electrical activity measured during delivery of pacing therapy using the plurality of different interventricular pacing delays using the selected left ventricular pacing vector. The computing apparatus may be further configured to determine whether the generated EHI from the electrical activity measured during the pacing therapy using the selected left ventricular pacing vector and the selected interventricular pacing delay indicates acceptable therapy, and in response to determination that the generated EHI from the electrical activity measured during the pacing therapy using the selected left ventricular pacing vector and the selected interventricular pacing delay does indicate acceptable therapy, provide an indication that the pacing therapy is complete. In response to determination that the generated EHI from the electrical activity measured during the pacing therapy using the selected left ventricular pacing vector and the selected interventricular pacing delay does not indicate acceptable therapy, the computing apparatus may be further configured to select one atrioventricular pacing delay of a plurality of different atrioventricular pacing delays based on the generated EHI from the electrical activity measured during delivery of pacing therapy using the plurality of different atrioventricular pacing delays using the selected left ventricular pacing vector and the selected interventricular pacing delay.

One illustrative method, e.g., for use in cardiac therapy configuration, may include obtaining external electrical activity from tissue of a patient and generating electrical heterogeneity information (EHI) based on the obtained electrical activity. The method may further include selecting a left ventricular pacing vector of a plurality of different left ventricular pacing vectors based on the generated EHI from the electrical activity measured during delivery of pacing therapy using the plurality of different left ventricular pacing vectors. Each different left ventricular pacing vector may use a combination of pacing electrodes different from the remaining different left ventricular pacing vectors. The method may further include selecting one of adaptive pacing therapy or non-adaptive pacing therapy based on the generated EHI from the electrical activity measured during delivery of adaptive pacing therapy using the selected left ventricular pacing vector. The adaptive pacing therapy may adjust one or more pacing timing intervals based on one or both of intrinsic atrioventricular conduction and heart rate. In response to selection of adaptive pacing therapy, the method may further include providing an indication that the pacing therapy is complete. In response to selection of non-adaptive pacing therapy, the method may further include selecting one interventricular pacing delay of a plurality of different interventricular pacing delays based on the generated EHI from the electrical activity measured during delivery of pacing therapy using the plurality of different interventricular pacing delays using the selected left ventricular pacing vector. The method may further include determining whether the generated EHI from the electrical activity measured during the pacing therapy using the selected left ventricular pacing vector and the selected interventricular pacing delay indicates acceptable therapy. In response to determination that the generated EHI from the electrical activity measured during the pacing therapy using the selected left ventricular pacing vector and the selected interventricular pacing delay does indicate acceptable therapy, the method may further include providing an indication that the pacing therapy is complete. In response to determination that the generated EHI from the electrical activity measured during the pacing therapy using the selected left ventricular pacing vector and the selected interventricular pacing delay does not indicate acceptable therapy, the method may further include selecting one atrioventricular pacing delay of a plurality of different atrioventricular pacing delays based on the generated EHI from the electrical activity measured during delivery of pacing therapy using the plurality of different atrioventricular pacing delays using the selected left ventricular pacing vector and the selected interventricular pacing delay.

One illustrative system, e.g., for use in cardiac therapy configuration, may include a computing apparatus. The computing apparatus may include processing circuitry and may be configured to obtain external electrical activity measured from tissue of a patient, generate electrical heterogeneity information (EHI) based on the obtained electrical activity, and based on the generated EHI from the electrical activity measured during delivery of pacing therapy, determine a left ventricular pacing vector of a plurality of different left ventricular pacing vectors and select one of adaptive pacing therapy or non-adaptive pacing therapy. The adaptive pacing therapy may adjust one or more pacing timing intervals based on one or both of intrinsic atrioventricular conduction and heart rate. The above summary is not intended to describe each embodiment or every implementation of the present disclosure. A more complete understanding will become apparent and appreciated by referring to the following detailed description and claims taken in conjunction with the accompanying drawings.

### BRIEF DESCRIPTION OF THE DRAWINGS

FIG. 1 is a diagram of an illustrative system including electrode apparatus, display apparatus, and computing apparatus.

FIGS. 2-3 are diagrams of illustrative external electrode apparatus for measuring torso-surface potentials.

FIG. 4 provides a block diagram of an illustrative method of evaluation and configuration of cardiac therapy.

FIGS. 5A-5B provide a block diagram of more detailed illustrative method of evaluation and configuration of cardiac therapy.

FIG. 6 is an illustrative graphical user interface (GUI) to be used with the methods of evaluation and configuration of cardiac therapy of FIGS. 4-5.

FIG. 7 is a diagram of an illustrative system including an illustrative implantable medical device (IMD).

FIG. 8A is a diagram of the illustrative IMD of FIG. 7.

FIG. 8B is a diagram of an enlarged view of a distal end of the electrical lead disposed in the left ventricle of FIG. 8A.

FIG. 9A is a block diagram of an illustrative IMD, e.g., of the systems of FIGS. 7-8.

FIG. 9B is another block diagram of an illustrative IMD (e.g., an implantable pulse generator) circuitry and associated leads employed in the systems of FIGS. 7-8).

### DETAILED DESCRIPTION OF ILLUSTRATIVE EMBODIMENTS

In the following detailed description of illustrative embodiments, reference is made to the accompanying figures of the drawing which form a part hereof, and in which are shown, by way of illustration, specific embodiments which may be practiced. It is to be understood that other embodiments may be utilized and structural changes may be made without departing from (e.g., still falling within) the scope of the disclosure presented hereby.

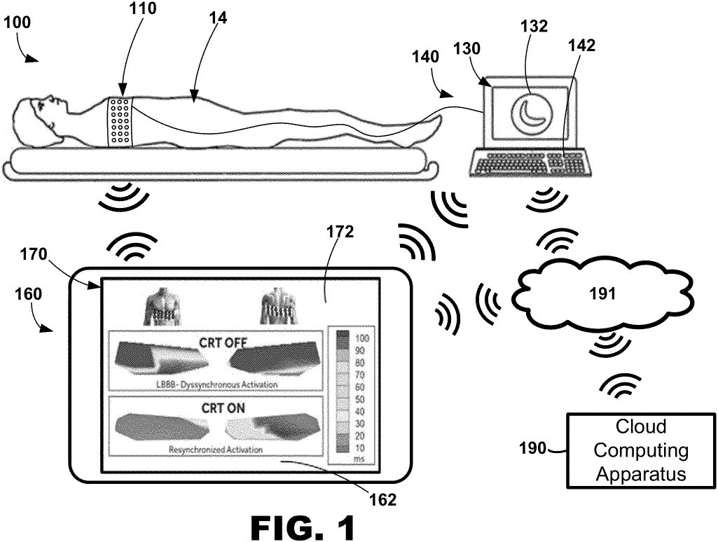

Illustrative systems, methods, and devices shall be described with reference to FIGS. 1-9. It will be apparent to one skilled in the art that elements or processes from one embodiment may be used in combination with elements or processes of the other embodiments, and that the possible embodiments of such systems, methods, and devices using combinations of features set forth herein is not limited to the specific embodiments shown in the Figures and/or described herein. Further, it will be recognized that the embodiments described herein may include many elements that are not necessarily shown to scale. Still further, it will be recognized that timing of the processes and the size and shape of various elements herein may be modified but still fall within the scope of the present disclosure, although certain timings, one or more shapes and/or sizes, or types of elements, may be advantageous over others.

A plurality of electrocardiogram (ECG) signals (e.g., torso-surface potentials) may be measured, or monitored, using a plurality of external electrodes positioned about the surface, or skin, of a patient. The ECG signals may be used to evaluate and configure cardiac therapy such as, e.g., cardiac therapy provide by an implantable medical device performing cardiac resynchronization therapy (CRT). As described herein, the ECG signals may be gathered or obtained noninvasively since, e.g., implantable electrodes may not be used to measure the ECG signals. Further, the ECG signals may be used to determine cardiac electrical activation times, which may be used to generate various metrics (e.g., electrical heterogeneity information) that may be used automatically by the illustrative system, device, or method and/or by a user (e.g., physician) to optimize one or more settings, or parameters, of cardiac therapy (e.g., pacing therapy) such as CRT (e.g., adaptive CRT or non-adaptive CRT).

Various illustrative systems, methods, devices, and graphical user interfaces provided thereby may be configured to use electrode apparatus including external electrodes, display apparatus, and computing apparatus to noninvasively assist a user (e.g., a physician) in the evaluation of cardiac health and/or the configuration (e.g., optimization) of cardiac therapy. An illustrative system 100 including electrode apparatus 110 and computing apparatus is depicted in FIG. 1. The computing apparatus may include, among other things, a local computing device 140, a remote computing device 160, and a cloud computing device 190. It is to be understood that the computing apparatus may include any one of the local computing device 140, the remote computing device 160, and the cloud computing device 190 or any combination of the local computing device 140, the remote computing device 160, and the cloud computing device 190 operating in conjunction with each other.

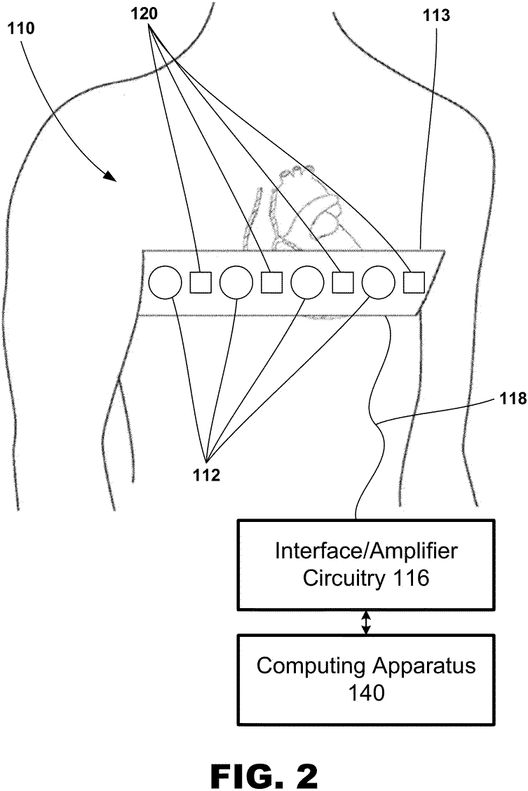

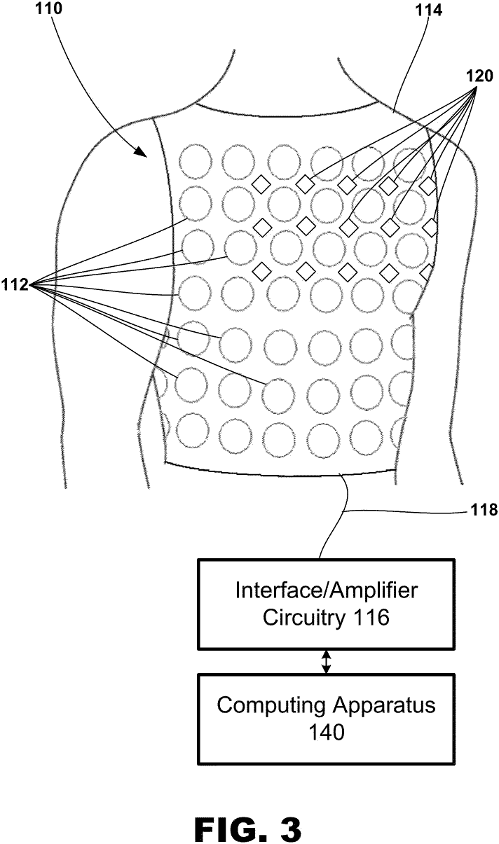

The electrode apparatus 110 as shown includes a plurality of electrodes incorporated, or included, within a band wrapped around the chest, or torso, of a patient 14. The electrode apparatus 110 is operatively coupled to the local computing device 140 (e.g., through one or wired electrical connections, wirelessly, etc.) to provide electrical signals from each of the electrodes to the local computing device 140 for analysis, evaluation, etc. Illustrative electrode apparatus may be described in U.S. Pat. No. 9,320,446 entitled “Bioelectric Sensor Device and Methods” filed Mar. 27, 2014, and issued on Mar. 26, 2016, which is incorporated herein by reference in its entirety. Further, illustrative electrode apparatus 110 will be described in more detail in reference to FIGS. 2-3.

Although not described herein, the illustrative system 100 may further include imaging apparatus. The imaging apparatus may be any type of imaging apparatus configured to image, or provide images of, at least a portion of the patient in a noninvasive manner. For example, the imaging apparatus may not use any components or parts that may be located within the patient to provide images of the patient except noninvasive tools such as contrast solution. It is to be understood that the illustrative systems, methods, and interfaces described herein may further use imaging apparatus to provide noninvasive assistance to a user (e.g., a physician) to locate, or place, one or more pacing electrodes proximate the patient’s heart in conjunction with the configuration of cardiac therapy.

For example, the illustrative systems, methods, and devices, may provide image guided navigation that may be used to navigate leads including electrodes, leadless electrodes, wireless electrodes, catheters, etc., within the patient’s body while also providing noninvasive cardiac therapy configuration including determination of an effective, or optimal, pre-excitation intervals such as A-V and V-V intervals, etc. Illustrative systems, methods, and devices that use imaging apparatus and/or electrode apparatus may be described in U.S. Pat. App. Pub. No. 2014/0371832 to Ghosh published on Dec. 18, 2014, U.S. Pat. App. Pub. No. 2014/0371833 to Ghosh et al. published on Dec. 18, 2014, U.S. Pat. App. Pub. No. 2014/0323892 to Ghosh et al. published on Oct. 30, 2014, U.S. Pat. App. Pub. No. 2014/0323882 to Ghosh et al. published on Oct. 20, 2014, each of which is incorporated herein by reference in its entirety.

Illustrative imaging apparatus may be configured to capture x-ray images and/or any other alternative imaging modality. For example, the imaging apparatus may be configured to capture images, or image data, using isocentric fluoroscopy, bi-plane fluoroscopy, ultrasound, computed tomography (CT), multi-slice computed tomography (MSCT), magnetic resonance imaging (MRI), high frequency ultrasound (HIFU), optical coherence tomography (OCT), intra-vascular ultrasound (IVUS), two dimensional (2D) ultrasound, three dimensional (3D) ultrasound, four dimensional (4D) ultrasound, intraoperative CT, intraoperative MRI, etc. Further, it is to be understood that the imaging apparatus may be configured to capture a plurality of consecutive images (e.g., continuously) to provide video frame data. In other words, a plurality of images taken over time using the imaging apparatus may provide video frame, or motion picture, data. An exemplary system that employs ultrasound can be found in U.S. Pat. App. Pub. No. 2017/0303840 to Stadler et al. published on Oct. 26, 2017, which is incorporated by reference in its entirety. Additionally, the images may also be obtained and displayed in two, three, or four dimensions. In more advanced forms, four-dimensional surface rendering of the heart or other regions of the body may also be achieved by incorporating heart data or other soft tissue data from a map or from pre-operative image data captured by MRI, CT, or echocardiography modalities. Image datasets from hybrid modalities, such as positron emission tomography (PET) combined with CT, or single photon emission computer tomography (SPECT) combined with CT, could also provide functional image data superimposed onto anatomical data, e.g., to be used to navigate implantable apparatus to target locations within the heart or other areas of interest.

Systems and/or imaging apparatus that may be used in conjunction with the illustrative systems, methods, and devices described herein are described in U.S. Pat. App. Pub. No. 2005/0008210 to Evron et al. published on Jan. 13, 2005, U.S. Pat. App. Pub. No. 2006/0074285 to Zarkh et al. published on Apr. 6, 2006, U.S. Pat. No. 8,731,642 to Zarkh et al. issued on May 20, 2014, U.S. Pat. No. 8,861,830 to Brada et al. issued on Oct. 14, 2014, U.S. Pat. No. 6,980,675 to Evron et al. issued on Dec. 27, 2005, U.S. Pat. No. 7,286,866 to Okerlund et al. issued on Oct. 23, 2007, U.S. Pat. No. 7,308,297 to Reddy et al. issued on Dec. 11, 2011, U.S. Pat. No. 7,308,299 to Burrell et al. issued on Dec. 11, 2011, U.S. Pat. No. 7,321,677 to Evron et al. issued on Jan. 22, 2008, U.S. Pat. No. 7,346,381 to Okerlund et al. issued on Mar. 18, 2008, U.S. Pat. No. 7,454,248 to Burrell et al. issued on Nov. 18, 2008, U.S. Pat. No. 7,499,743 to Vass et al. issued on Mar. 3, 2009, U.S. Pat. No. 7,565,190 to Okerlund et al. issued on Jul. 21, 2009, U.S. Pat. No. 7,587,074 to Zarkh et al. issued on Sep. 8, 2009, U.S. Pat. No. 7,599,730 to Hunter et al. issued on Oct. 6, 2009, U.S. Pat. No. 7,613,500 to Vass et al. issued on Nov. 3, 2009, U.S. Pat. No. 7,742,629 to Zarkh et al. issued on Jun. 22, 2010, U.S. Pat. No. 7,747,047 to Okerlund et al. issued on Jun. 29, 2010, U.S. Pat. No. 7,778,685 to Evron et al. issued on Aug. 17, 2010, U.S. Pat. No. 7,778,686 to Vass et al. issued on Aug. 17, 2010, U.S. Pat. No. 7,813,785 to Okerlund et al. issued on Oct. 12, 2010, U.S. Pat. No. 7,996,063 to Vass et al. issued on Aug. 9, 2011, U.S. Pat. No. 8,060,185 to Hunter et al. issued on Nov. 15, 2011, and U.S. Pat. No. 8,401,616 to Verard et al. issued on Mar. 19, 2013, each of which is incorporated herein by reference in its entirety.

The local computing device 140, the remote computing device 160, and the cloud computing device 190 may be configured to monitor (e.g., using the electrode apparatus), generate, and analyze data such as, e.g., electrical signals (e.g., electrocardiogram data), electrical activation times, electrical heterogeneity information, etc. For example, one cardiac cycle, or one heartbeat, of a plurality of cardiac cycles, or heartbeats, represented by the electrical signals collected or monitored by the electrode apparatus 110 may be analyzed and evaluated for one or more metrics including activation times and electrical heterogeneity information that may be pertinent to the therapeutic nature of one or more parameters related to cardiac therapy such as, e.g., pacing parameters, pacing mode, pacing vector, lead location, etc. More specifically, for example, the QRS complex of a single cardiac cycle may be evaluated for one or more metrics such as, e.g., QRS onset, QRS offset, QRS peak, electrical activation times referenced to earliest activation time, electrical heterogeneity information (EHI) such as left ventricular or thoracic standard deviation of electrical activation times (LVED), standard deviation of activation times (SDAT), and average left ventricular or thoracic surrogate electrical activation times (LVAT), QRS duration (e.g., interval between QRS onset to QRS offset), difference between average left surrogate and average right surrogate activation times, relative or absolute QRS morphology, difference between a higher percentile and a lower percentile of activation times (higher percentile may be 90%, 80%, 75%, 70%, etc. and lower percentile may be 10%, 15%, 20%, 25% and 30%, etc.), other statistical measures of central tendency (e.g., median or mode), dispersion (e.g., mean deviation, standard deviation, variance, interquartile deviations, range, etc.), etc. Further, each of the one or more metrics may be location specific. For example, some metrics may be computed from signals recorded, or monitored, from electrodes positioned about a selected area of the patient such as, e.g., the left side of the patient, the right side of the patient, etc. Additionally, the local computing device 140 and the remote computing device 160 may each include display apparatus 130, 170, respectively, that may be configured to display such data.

In at least one embodiment, each of the local computing device 140, the remote computing device 160, and the cloud computing device 190 may be a server, a personal computer, a tablet computer, a mobile device, and a cellular telephone. The local computing device 140 may be configured to receive input from input apparatus 142 (e.g., a keyboard) and transmit output to the display apparatus 130, and the remote computing device 160 may be configured to receive input from input apparatus 162 (e.g., a touchscreen) and transmit output to the display apparatus 170. The local computing device 140, the remote computing device 160, and the cloud computing device 190 may include data storage that may allow for access to processing programs or routines and/or one or more other types of data, e.g., for analyzing a plurality of electrical signals captured by the electrode apparatus 110, for determining QRS onsets, QRS offsets, medians, modes, averages, peaks or maximum values, valleys or minimum values in such electrical signals, for determining electrical activation times, for configuring one or more pacing parameters, or settings, such as, e.g., pacing rate, ventricular pacing rate, A-V interval, V-V interval, pacing pulse width, pacing vector, multipoint pacing vector (e.g., left ventricular vector quad lead), pacing voltage, pacing configuration (e.g., biventricular pacing, right ventricle only pacing, left ventricle only pacing, etc.), for driving graphical user interfaces used to configured one or more pacing parameters, for arrhythmia detection and treatment, for configuring rate adaptive settings, etc.

The local computing device 140 may be operatively coupled to the input apparatus 142 and the display apparatus 130 to, e.g., transmit data to and from each of the input apparatus 142 and the display apparatus 130, and the remote computing device 160 may be operatively coupled to the input apparatus 162 and the display apparatus 170 to, e.g., transmit data to and from each of the input apparatus 162 and the display apparatus 170. For example, the local computing device 140 and the remote computing device 160 may be electrically coupled to the input apparatus 142, 162 and the display apparatus 130, 170 using, e.g., analog electrical connections, digital electrical connections, wireless connections, bus-based connections, network-based connections, internet-based connections, etc. As described further herein, a user may provide input to the input apparatus 142, 162 to view and/or select one or more pieces of configuration information related to the cardiac therapy delivered by cardiac therapy apparatus such as, e.g., an implantable medical device.

Although as depicted the input apparatus 142 is a keyboard and the input apparatus 162 is a touchscreen, it is to be understood that the input apparatus 142, 162 may include any apparatus capable of providing input to the local computing device 140 and the computing device 160 to perform the functionality, methods, and/or logic described herein. For example, the input apparatus 142, 162 may include a keyboard, a mouse, a trackball, a touchscreen (e.g., capacitive touchscreen, a resistive touchscreen, a multi-touch touchscreen, etc.), etc. Likewise, the display apparatus 130, 170 may include any apparatus capable of displaying information to a user, such as a graphical user interface 132, 172 including electrode status information, graphical maps of electrical activation, a plurality of signals for the external electrodes over one or more heartbeats, QRS complexes, various pacing parameters (e.g., including selected pacing parameters as determined using the illustrative systems, methods, and devices described herein, electrical heterogeneity information (EHI), textual instructions, graphical depictions of anatomy of a human heart, images or graphical depictions of the patient’s heart, graphical depictions of locations of one or more electrodes, graphical depictions of a human torso, images or graphical depictions of the patient’s torso, graphical depictions or actual images of implanted electrodes and/or leads, etc. Further, the display apparatus 130, 170 may include a liquid crystal display, an organic light-emitting diode screen, a touchscreen, a cathode ray tube display, etc.

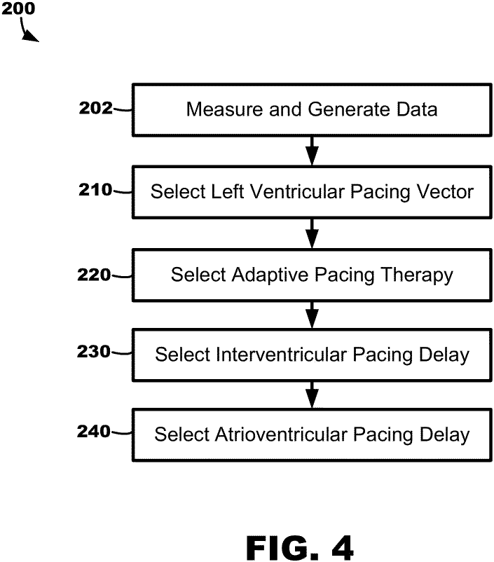

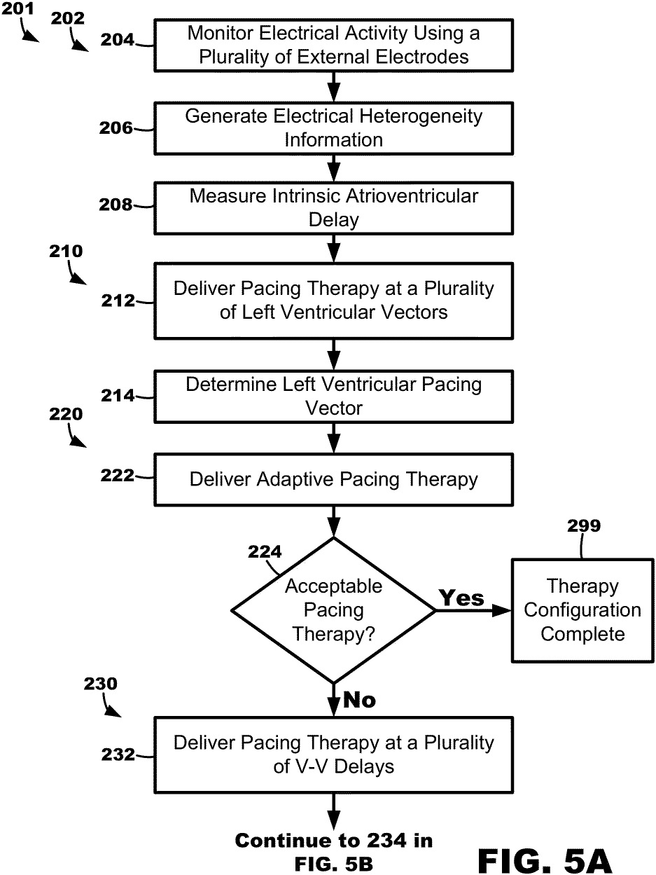

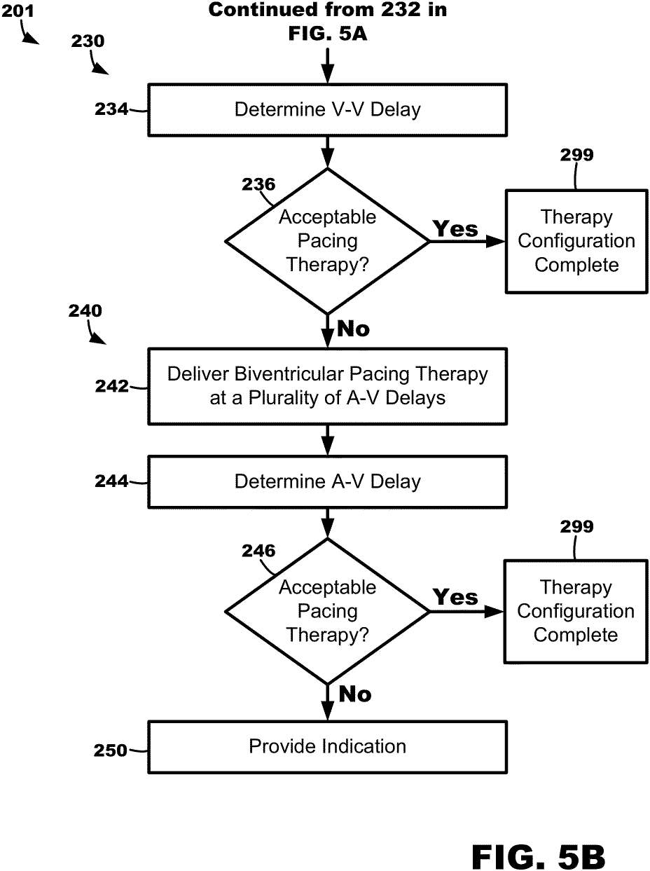

The processing programs or routines stored and/or executed by the local computing device 140, the remote computing device 160, and the cloud computing device 190 may include programs or routines for computational mathematics, matrix mathematics, decomposition algorithms, compression algorithms (e.g., data compression algorithms), calibration algorithms, image construction algorithms, signal processing algorithms (e.g., various filtering algorithms, Fourier transforms, fast Fourier transforms, etc.), standardization algorithms, comparison algorithms, vector mathematics, or any other processing used to implement one or more illustrative methods and/or processes described herein. Data stored and/or used by the local computing device 140, the remote computing device 160, and the cloud computing device 190 may include, for example, electrical signal/waveform data from the electrode apparatus 110 (e.g., a plurality of QRS complexes), electrical activation times from the electrode apparatus 110, cardiac sound/signal/waveform data from acoustic sensors, graphics (e.g., graphical elements, icons, buttons, windows, dialogs, pull-down menus, graphic areas, graphic regions, 3D graphics, etc.), graphical user interfaces, results from one or more processing programs or routines employed according to the disclosure herein (e.g., electrical signals, electrical heterogeneity information, etc.), or any other data that may be used for carrying out the one and/or more processes or methods described herein. In one or more embodiments, one or more of the local computing device 140, the remote computing device 160, and the cloud computing device 190 may be configured to select a left ventricular pacing vector, select adaptive or non-adaptive pacing therapy, select an interventricular pacing delay, and a select an atrioventricular pacing delay based on monitored and generated data from, for example, the electrode apparatus 110 as will be described further herein with respect to FIGS. 4-5. In particular, for example, one or more of the local computing device 140, the remote computing device 160, and the cloud computing device 190 may monitor electrical activity using the electrode apparatus 110 and generate electrical heterogeneity information (EHI) therefrom during delivery of pacing therapy a plurality of different configurations or settings. For instance, one or more of the local computing device 140, the remote computing device 160, and the cloud computing device 190 may generate EHI from electrical activity measured during delivery of pacing therapy at a plurality of different left ventricular pacing vectors, and then may determine which of the plurality of different left ventricular pacing vectors is desired (e.g., optimal), which may be selected, as will be described further herein with respect to FIG. 5A. Further, one or more of the local computing device 140, the remote computing device 160, and the cloud computing device 190 may generate EHI from electrical activity measured during delivery of pacing therapy configured as adaptive pacing therapy, and then may determine whether adaptive pacing therapy is desired (e.g., optimal), which may be selected, as will be described further herein with respect to FIG. 5A. Still further, one or more of the local computing device 140, the remote computing device 160, and the cloud computing device 190 may generate EHI from electrical activity measured during delivery of pacing therapy at a plurality of different interventricular pacing delays, and then may determine which of the plurality of different interventricular pacing delays is desired (e.g., optimal), which may be selected, as will be described further herein with respect to FIG. 5B. And still further, one or more of the local computing device 140, the remote computing device 160, and the cloud computing device 190 may generate EHI from electrical activity measured during delivery of pacing therapy at a plurality of different atrioventricular pacing delays, and then may determine which of the plurality of different atrioventricular pacing delays is desired (e.g., optimal), which may be selected, as will be described further herein with respect to FIG. 5B.

In one or more embodiments, the illustrative systems, methods, devices, and interfaces may be implemented using one or more computer programs executed on programmable computers, such as computers that include, for example, processing capabilities, data storage (e.g., volatile or non-volatile memory and/or storage elements), input devices, and output devices. Program code and/or logic described herein may be applied to input data to perform functionality described herein and generate desired output information. The output information may be applied as input to one or more other systems, methods, devices, and/or interfaces as described herein or as would be applied in a known fashion.

The one or more programs used to implement the systems, methods, devices, and/or interfaces described herein may be provided using any programmable language, e.g., a high-level procedural and/or object orientated programming language that is suitable for communicating with a computer. Any such programs may, for example, be stored on any suitable device, e.g., a storage media, that is readable by a general or special purpose program running on a computer (e.g., including processing circuitry or processing apparatus) for configuring and operating the computer when the suitable device is read for performing the procedures described herein. In other words, at least in one embodiment, the illustrative systems, methods, devices, and interfaces may be implemented using a tangible computer readable storage medium, configured with a computer program, where the storage medium so configured causes the computer to operate in a specific and predefined manner to perform functions described herein. Further, in at least one embodiment, the illustrative systems, methods, devices, and interfaces may be described as being implemented by logic (e.g., object code) encoded in one or more non-transitory media that includes code for execution and, when executed by a processor or processing circuitry, is operable to perform operations such as the methods, processes, and/or functionality described herein.

The local computing device 140, the remote computing device 160, and the cloud computing device 190 may be, for example, any fixed or mobile computer system (e.g., a controller, a microcontroller, a personal computer, minicomputer, tablet computer, etc.) including processing circuitry. The exact configurations of the local computing device 140, the remote computing device 160, and the cloud computing device 190 are not limiting, and essentially any device including processing circuitry and capable of providing suitable computing capabilities and control capabilities (e.g., signal analysis, mathematical functions such as medians, modes, averages, maximum value determination, minimum value determination, slope determination, minimum slope determination, maximum slope determination, graphics processing, etc.) may be used. As described herein, a digital file may be any medium (e.g., volatile or non-volatile memory, a CD-ROM, a punch card, magnetic recordable tape, etc.) containing digital bits (e.g., encoded in binary, trinary, etc.) that may be readable and/or writeable by the local computing device 140, the remote computing device 160, and the cloud computing device 190 described herein. Also, as described herein, a file in user-readable format may be any representation of data (e.g., ASCII text, binary numbers, hexadecimal numbers, decimal numbers, graphically, etc.) presentable on any medium (e.g., paper, a display, etc.) readable and/or understandable by a user.

In view of the above, it will be readily apparent that the functionality as described in one or more embodiments according to the present disclosure may be implemented in any manner as would be known to one skilled in the art. As such, the computer language, the computer system, or any other software/hardware which is to be used to implement the processes described herein shall not be limiting on the scope of the systems, processes, or programs (e.g., the functionality provided by such systems, processes, or programs) described herein.

The illustrative electrode apparatus 110 may be configured to measure body-surface potentials of a patient 14 and, more particularly, torso-surface potentials of a patient 14. As shown in FIG. 2, the illustrative electrode apparatus 110 may include a set, or array, of external electrodes 112, a strap 113, and interface/amplifier circuitry 116. The electrodes 112 may be attached, or coupled, to the strap 113 and the strap 113 may be configured to be wrapped around the torso of a patient 14 such that the electrodes 112 surround the patient’s heart. As further illustrated, the electrodes 112 may be positioned around the circumference of a patient 14, including the posterior, lateral, posterolateral, anterolateral, and anterior locations of the torso of a patient 14.

The illustrative electrode apparatus 110 may be further configured to measure, or monitor, sounds from the patient 14. As shown in FIG. 2, the illustrative electrode apparatus 110 may include a set, or array, of acoustic sensors 120 attached, or coupled, to the strap 113. The strap 113 may be configured to be wrapped around the torso of a patient 14 such that the acoustic sensors 120 surround the patient’s heart. As further illustrated, the acoustic sensors 120 may be positioned around the circumference of a patient 14, including the posterior, lateral, posterolateral, anterolateral, and anterior locations of the torso of a patient 14.

Further, the electrodes 112 and the acoustic sensors 120 may be electrically connected to interface/amplifier circuitry 116 via wired connection 118. The interface/amplifier circuitry 116 may be configured to amplify the signals from the electrodes 112 and the acoustic sensors 120 and provide the signals to one or more of the local computing device 140, the remote computing device 160, and the cloud computing device 190. Other illustrative systems may use a wireless connection to transmit the signals sensed by electrodes 112 and the acoustic sensors 120 to the interface/amplifier circuitry 116 and, in turn, to one or more of the local computing device 140, the remote computing device 160, and the cloud computing device 190, e.g., as channels of data. In one or more embodiments, the interface/amplifier circuitry 116 may be electrically coupled to the local computing device 140 using, e.g., analog electrical connections, digital electrical connections, wireless connections, bus-based connections, network-based connections, internet-based connections, etc.

Although in the example of FIG. 2 the electrode apparatus 110 includes a strap 113, in other examples any of a variety of mechanisms, e.g., tape or adhesives, may be employed to aid in the spacing and placement of electrodes 112 and the acoustic sensors 120. In some examples, the strap 113 may include an elastic band, strip of tape, or cloth. Further, in some examples, the strap 113 may be part of, or integrated with, a piece of clothing such as, e.g., a t-shirt or hospital gown. In other examples, the electrodes 112 and the acoustic sensors 120 may be placed individually on the torso of a patient 14. Further, in other examples, one or both of the electrodes 112 (e.g., arranged in an array) and the acoustic sensors 120 (e.g., also arranged in an array) may be part of, or located within, patches, vests, and/or other manners of securing the electrodes 112 and the acoustic sensors 120 to the torso of the patient 14. Still further, in other examples, one or both of the electrodes 112 and the acoustic sensors 120 may be part of, or located within, two sections of material or two patches. One of the two patches may be located on the anterior side of the torso of the patient 14 (to, e.g., monitor electrical signals representative of the anterior side of the patient’s heart, measure surrogate cardiac electrical activation times representative of the anterior side of the patient’s heart, monitor or measure sounds of the anterior side of the patient, etc.) and the other patch may be located on the posterior side of the torso of the patient 14 (to, e.g., monitor electrical signals representative of the posterior side of the patient’s heart, measure surrogate cardiac electrical activation times representative of the posterior side of the patient’s heart, monitor or measure sounds of the posterior side of the patient, etc.). And still further, in other examples, one or both of the electrodes 112 and the acoustic sensors 120 may be arranged in a top row and bottom row that extend from the anterior side of the patient 14 across the left side of the patient 14 to the posterior side of the patient 14. Yet still further, in other examples, one or both of the electrodes 112 and the acoustic sensors 120 may be arranged in a curve around the armpit area and may have an electrode/sensor-density that less dense on the right thorax that the other remaining areas.

The electrodes 112 may be configured to surround the heart of the patient 14 and record, or monitor, the electrical signals associated with the depolarization and repolarization of the heart after the signals have propagated through the torso of a patient 14. Each of the electrodes 112 may be used in a unipolar configuration to sense the torso-surface potentials that reflect the cardiac signals. The interface/amplifier circuitry 116 may also be coupled to a return or indifferent electrode (not shown) that may be used in combination with each electrode 112 for unipolar sensing.

In some examples, there may be about 12 to about 40 electrodes 112 and about 12 to about 40 acoustic sensors 120 spatially distributed around the torso of a patient. Other configurations may have more or fewer electrodes 112 and more or fewer acoustic sensors 120. For example, the number of electrodes 112 of the electrode apparatus 110 may be greater than or equal to 10, greater than or equal to 12, greater than or equal to 15, greater than or equal to 20, greater than or equal to 35, greater than or equal to 40, etc. and/or less than or equal to 80 electrodes, less than or equal to 70 electrodes, less than or equal to 60 electrodes, less than or equal to 50 electrodes, less than or equal to 45 electrodes, less than or equal to 38 electrodes, etc. In at least one embodiment, the electrode apparatus 110 includes 40 electrodes 112 with 20 of the electrodes 112 configured to be located on the posterior of the patient and the 20 of the electrodes 112 configured to be located on the anterior of the patient. In some embodiment, more electrodes 112 may be configured to be positioned on the anterior of the patient than the posterior of the patient or more electrodes 112 may be configured to be positioned on the posterior of the patient than the anterior of the patient. For example, in one embodiment, 25 electrodes 112 may be configured to be positioned on the anterior of the patient and 15 electrodes 112 may be configured to be positioned on the posterior of the patient. Additionally, in one embodiment, the electrodes 112 may be included as part of a 12-lead ECG apparatus. It is to be understood that the electrodes 112 and acoustic sensors 120 may not be arranged or distributed in an array extending all the way around or completely around the patient 14. Instead, the electrodes 112 and acoustic sensors 120 may be arranged in an array that extends only part of the way or partially around the patient 14. For example, the electrodes 112 and acoustic sensors 120 may be distributed on the anterior, posterior, and left sides of the patient with less or no electrodes and acoustic sensors proximate the right side (including posterior and anterior regions of the right side of the patient).

The local computing device 140 may record and analyze the torso-surface potential signals sensed by electrodes 112 and the sound signals sensed by the acoustic sensors 120, which are amplified/conditioned by the interface/amplifier circuitry 116. The local computing device 140 may be configured to analyze the electrical signals from the electrodes 112 to provide electrocardiogram (ECG) signals, information, or data from the patient’s heart as will be further described herein. The local computing device 140 may be configured to analyze the electrical signals from the acoustic sensors 120 to provide sound signals, information, or data from the patient’s body and/or devices implanted therein (such as a left ventricular assist device).

Additionally, the local computing device 140, the remote computing device 160, and the cloud computing device 190 may be configured to provide graphical user interfaces 132, 172 depicting various information related to the electrode apparatus 110 and the data gathered, or sensed, using the electrode apparatus 110. In at least one embodiment, the cloud computing device 190 may provide one or both of the graphical user interfaces 132, 172 to the local computing device 140 and the remote computing device 160. For example, the graphical user interfaces 132, 172 may depict ECGs including QRS complexes obtained using the electrode apparatus 110 and sound data including sound waves obtained using the acoustic sensors 120 as well as other information related thereto. Illustrative systems, methods, and devices may noninvasively use the electrical information collected using the electrode apparatus 110 and the sound information collected using the acoustic sensors 120 to evaluate a patient’s cardiac health and to evaluate and configure cardiac therapy being delivered to the patient.

Further, the electrode apparatus 110 may further include reference electrodes and/or drive electrodes to be, e.g., positioned about the lower torso of the patient 14, that may be further used by the system 100. For example, the electrode apparatus 110 may include three reference electrodes, and the signals from the three reference electrodes may be combined to provide a reference signal. Further, the electrode apparatus 110 may use three caudal reference electrodes (e.g., instead of standard references used in a Wilson Central Terminal) to get a “true” unipolar signal with less noise from averaging three caudally located reference signals.

FIG. 3 illustrates another illustrative electrode apparatus 110 that includes a plurality of electrodes 112 configured to surround the heart of the patient 14 and record, or monitor, the electrical signals associated with the depolarization and repolarization of the heart after the signals have propagated through the torso of the patient 14 and a plurality of acoustic sensors 120 configured to surround the heart of the patient 14 and record, or monitor, the sound signals associated with the heart after the signals have propagated through the torso of the patient 14. The electrode apparatus 110 may include a vest 114 upon which the plurality of electrodes 112 and the plurality of acoustic sensors 120 may be attached, or to which the electrodes 112 and the acoustic sensors 120 may be coupled. In at least one embodiment, the plurality, or array, of electrodes 112 may be used to collect electrical information such as, e.g., surrogate electrical activation times. Similar to the electrode apparatus 110 of FIG. 2, the electrode apparatus 110 of FIG. 3 may include interface/amplifier circuitry 116 electrically coupled to each of the electrodes 112 and the acoustic sensors 120 through a wired connection 118 and be configured to transmit signals from the electrodes 112 and the acoustic sensors 120 to the local computing device 140. As illustrated, the electrodes 112 and the acoustic sensors 120 may be distributed over the torso of a patient 14, including, for example, the posterior, lateral, posterolateral, anterolateral, and anterior locations of the torso of a patient 14.

The vest 114 may be formed of fabric with the electrodes 112 and the acoustic sensors 120 attached to the fabric. The vest 114 may be configured to maintain the position and spacing of electrodes 112 and the acoustic sensors 120 on the torso of the patient 14. Further, the vest 114 may be marked to assist in determining the location of the electrodes 112 and the acoustic sensors 120 on the surface of the torso of the patient 14. In some examples, there may be about 25 to about 256 electrodes 112 and about 25 to about 256 acoustic sensors 120 distributed around the torso of the patient 14, though other configurations may have more or fewer electrodes 112 and more or fewer acoustic sensors 120.

The illustrative systems, methods, devices, and methods may be used to provide noninvasive assistance to configure of cardiac therapy being presently delivered to the patient (e.g., by an implantable medical device, etc.). For example, the illustrative systems, methods, and devices may be used to configure and/or adjust of one or more cardiac therapy settings such as, e.g., selection of pacing electrodes, selection of pacing vectors, selection of adaptive pacing therapy (e.g., adaptive cardiac resynchronization therapy) or non-adaptive pacing therapy (e.g., non-adaptive, or standard, cardiac resynchronization therapy), selection of optimized of atrioventricular (A-V) pacing delay, or interval and/or interventricular (V-V) pacing delay, or interval, of pacing therapy (e.g., left ventricular-only pacing therapy, left univentricular pacing therapy, biventricular pacing therapy, etc.).

Further, it is to be understood that the computing apparatus, e.g., include the local computing device 140, the remote computing device 160, and the cloud computing device 190, may be operatively coupled in a plurality of different ways so as to perform, or execute, the functionality described herein. For example, in the embodiment depicted, the computing device 140 may be wireless operably coupled to the remote computing device 160 as depicted by the wireless signal lines emanating therebetween. Additionally, as opposed to wireless connections, one or more of the local computing device 140 and the remoting computing device 160 may be operably coupled through one or wired electrical connections. Furthermore, one or both of the local computing device 140 and the remote computing device 160 may be operably coupled (e.g., wirelessly operably coupled, partially-wirelessly coupled, etc.) to the cloud computing device 190 via a network 191 such as, e.g., the internet. In one embodiment, the cloud computing device 190 may perform all or much of the data analysis on the data from the electrode apparatus 110 and output such analysis to one or both of the local computing device 140 and the remote computing device 160. It is be understood that the data processing and analysis described herein may be provide by one or more of the local computing device 140, the remote computing device 160, and the cloud computing device 190. In other words, the processing and analysis may be distributed among the computing apparatus such as the local computing device 140, the remote computing device 160, and the cloud computing device 190. Additionally, the cloud computing device 190 may include, among other things, an electronic medical records (EMR) system or database that may store information from the systems, methods, and apparatus described herein and provide information to such systems, methods, and apparatus. For example, the EMR of the cloud computing device 190 may provide images (e.g., CT images of the patient’s heart) to local computing device 140 and the remote computing device 160 to be used with the data provided by the electrode apparatus 110.

An illustrative method 200 of configuration of cardiac therapy is depicted in FIG. 4. The illustrative method 200 may be generally described to be used in the efficient, noninvasive configuration (e.g., optimization) of pacing therapy. The illustrative method 200 may be described as being noninvasive because the method does not use invasive apparatus to perform the evaluation of the pacing therapy. The pacing therapy being delivered, however, may be described as being invasive such as, e.g., one or more pacing electrodes may be implanted proximate a patient’s heart. Thus, the illustrative method 200 may be used to evaluate and configure such invasive cardiac therapy.

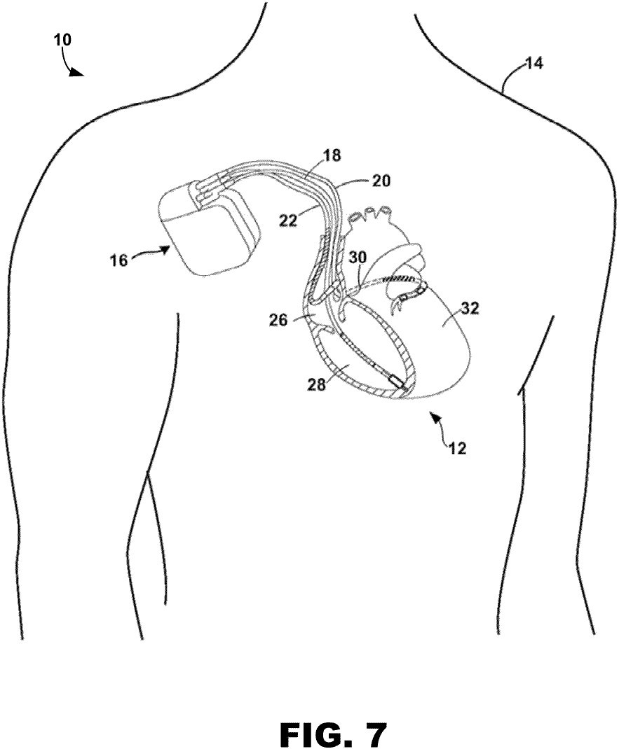

The illustrative method 200 may be generally described as sequence of steps to be implemented in a workflow “wizard” in a stand-alone illustrative system or device or in an integrated system or device solution that may be based on a minimum number of tests to get to a patient specific optimal pacing configuration using signals measured using external electrode apparatus. The pacing may be performed, or delivered by, a IMD such as, e.g., described herein with respect to FIGS. 7-9, while the method 200 may utilize the external electrode apparatus to measure, or monitor, data that may be used in the tests to obtain a patient specific optimal pacing configuration. Additionally, although the illustrative method described herein describes the use of data gathered, or obtained, from the external electrode apparatus, it is to be understood that data may also be obtained from the IMD delivering the therapy, and such data from the IMD may be used in conjunction with the data measured, or monitored, by the external electrode apparatus. The patient specific optimal pacing configuration may include one or more of a selected left ventricular pacing vector, a selected pacing mode of either adaptive pacing therapy or non-adaptive pacing therapy, a selected interventricular (VV) pacing delay, and a selected atrioventricular (AV) pacing delay for use in cardiac pacing therapy such as, e.g., left ventricular-only pacing therapy, biventricular pacing therapy, etc.

It is to be understood that pacing therapy may have a plurality of different pacing parameters such as e.g., AV pacing delay or timing, VV pacing delay or timing, selection of pacing electrodes or pacing vector (e.g., multisite pacing), pacing pulse widths, pacing pulse amplitudes (e.g., voltage), ventricular pacing rates, pacing configurations (e.g., biventricular pacing, right ventricle only pacing, left ventricle only pacing, etc.), adaptive or non-adaptive pacing therapy modes, rate adaptive pacing settings, etc. Each of these different pacing parameters may include a plurality of different settings or values. Some of the different pacing parameters may extend across an entire range of values.

The illustrative method 200 may be configured to evaluate, or test, each of the plurality of different pacing parameters such as pacing vector, pacing mode (e.g., adaptive on non-adaptive pacing therapy), VV pacing delay, and AV pacing delay. To do so, the illustrative method 200 may first, and throughout the remainder of the method 200, measure and generate data 202 that may be used to evaluate, or test, each of the plurality of different pacing parameters. In particular, the electrical activity measured using a plurality of external electrodes may be monitored, or measured, throughout method 200, which may be used to generate one or more metrics such as, e.g., electrical heterogeneity information (EHI). One illustrative example of measuring and generating data 202 will be described herein more specifically with respect to FIGS. 5A-5B.

The illustrative method 200 may then select a left ventricular pacing vector 210 of a plurality of different left ventricular pacing vectors based on the measured and generated data. Generally, data may be measured during delivery of pacing therapy using a plurality of different left ventricular pacing vectors, and the measured data and/or metrics derived therefrom may be evaluated to select the optimal left ventricular pacing vector. The ventricular pacing vector may be described as the identity of the one or more pacing electrodes used to deliver pacing to the left ventricle of a patient’s heart. A plurality of left ventricular pacing vectors may depend on the number of pacing electrodes positioned proximate the patient’s heart to delivering cardiac pacing therapy to the left ventricle. The plurality of left ventricular pacing vectors may include every different combination using one or more of the pacing electrodes positioned in or about the patient’s heart. For instance, if there are four pacing electrodes configured to pace to the left ventricle of the patient’s heart, sixteen different pacing vectors may be derived from the four pacing electrodes. In other words, sixteen different combinations of pacing electrodes may be used to pace the left ventricle of the patient’s heart when there are four pacing electrodes. One illustrative example of selecting a left ventricular pacing vector 210 will be described herein more specifically with respect to FIGS. 5A-5B.

The illustrative method 200 may then select one of adaptive pacing therapy or non-adaptive pacing therapy 220 based on the measured and generated data. Generally, data may be measured during delivery of adaptive pacing therapy using the selected left ventricular pacing vector, and the measured data and/or metrics derived therefrom may be evaluated to select whether adaptive pacing therapy or non-adaptive pacing therapy is optimal, and thus, should be utilized. As described herein, adaptive pacing therapy is configured to adjust pacing therapy configurations (e.g., biventricular pacing therapy, left ventricular only pacing therapy, etc.) based on one or both of intrinsic atrioventricular conduction and heart rate during delivery of the adaptive pacing therapy. In this way, adaptive pacing therapy may be described as adapting to the current condition of the patient. Non-adaptive pacing therapy may not change or adapt the pacing configuration. Instead, non-adaptive pacing therapy may include a fixed pacing mode or configuration that does not change. One illustrative example of selecting adaptive cardiac therapy or non-adaptive cardiac therapy 220 will be described herein more specifically with respect to FIGS. 5A-5B.

If adaptive pacing therapy is not selected 220, the illustrative method 200 may select a VV pacing delay 230 and optionally select an AV pacing delay 240. VV pacing delay (or timing) is the time period between a left ventricular pace and a right ventricular pace. The VV pacing delay may be described in terms of a time value. For instance, the VV pacing delay may be between about 5 milliseconds (ms) and about 50 ms. AV pacing delay (or timing) is the time period between intrinsic or paced depolarization of the atria and the delivery of a ventricular pace (e.g., left ventricular pace). The AV pacing delay may be described in terms of a time value. For instance, the AV pacing delay may be between about 40 ms and about 300 ms. The AV pacing delay may also be described in terms of a percentage of the patient’s intrinsic AV timing. For instance, the AV pacing delay may be between about 30% and about 90% of the patient’s intrinsic AV timing.

Generally, data may be measured during delivery of pacing therapy using the selected left ventricular pacing vector and a plurality of VV pacing delays, and the measured data and/or metrics derived therefrom may be evaluated to select the optimal VV pacing delay 230. Subsequently, data may be measured during delivery of pacing therapy using the selected left ventricular pacing vector, the selected VV pacing delay, and a plurality of AV pacing delays, and the measured data and/or metrics derived therefrom may be evaluated to select the optimal AV pacing delay 240. Illustrative examples of selecting VV pacing delay 230 and selecting AV pacing delay 240 will be described herein more specifically with respect to FIGS. 5A-5B.

A more detailed illustrative method of evaluation and configuration of cardiac therapy 201 is depicted in FIGS. 5A-5B. The method 201 includes the same or similar steps or processes as method 200 of FIG. 4 but described in a more detailed manner. For example, the method 200 includes measuring and generating data 202, selecting a left ventricular pacing vector 210, selecting adaptive or non-adaptive pacing therapy 220, selecting a VV pacing delay 230, and selecting an AV pacing delay 240.

The measuring and generating data 202 of method 201 includes monitoring, or measuring, electrical activity using a plurality of external electrodes 204. The plurality of external electrodes may be similar to the external electrodes provided by the electrode apparatus 110 as described herein with respect to FIGS. 1-3. For example, the plurality of external electrodes may be part, or incorporated into, a vest or band that is located about a patient’s torso. More specifically, the plurality of electrodes may be described as being surface electrodes positioned in an array configured to be located proximate the skin of the torso of a patient. For instance, each of the electrodes may be positioned or located about the torso of the patient so as to monitor electrical activity (e.g., acquire torso-potentials) from a plurality of different locations about the torso of the patient. Each of the different locations where the electrodes are located may correspond to the electrical activation of different portions or regions of cardiac tissue of the patient’s heart. Thus, for example, the plurality of electrodes may record, or monitor, the electrical signals associated with the depolarization and repolarization of a plurality of different locations of, or about, the heart after the signals have propagated through the torso of a patient. According to various embodiments, the plurality of external electrodes may include, or comprise, a plurality of anterior electrodes that are located proximate skin of the anterior of the patient’s torso, left lateral or left side electrodes that are located proximate skin of the left lateral or left side of the patient’s torso, and posterior electrodes that are located proximate skin of the posterior of the patient’s torso.

It may be described that, when using a plurality of external electrodes, the monitoring process 204 may provide a plurality of electrocardiograms (ECGs), signals representative of the depolarization and repolarization of the patient’s heart. The plurality of ECGs may, in turn, be used to generate surrogate cardiac electrical activation times representative of the depolarization of the heart. As described herein, surrogate cardiac electrical activation times may be, for example, representative of actual, or local, electrical activation times of one or more regions of the patient’s heart. Measurement of activation times can be performed by picking an appropriate fiducial point (e.g., peak values, minimum values, minimum slopes, maximum slopes, zero crossings, threshold crossings, etc. of a near or far-field EGM) and measuring time between the onset of cardiac depolarization (e.g., onset of QRS complexes) and the appropriate fiducial point (e.g., within the electrical activity). The activation time between the onset of the QRS complex (or the peak Q wave) to the fiducial point may be referred to as q-LV time. In at least one embodiment, the earliest QRS onset from all of the plurality of electrodes may be utilized as the starting point for each activation time for each electrode, and the maximum slope following the onset of the QRS complex may be utilized as the end point of each activation time for each electrode. The electrical activity measured, or monitored, that is prior to the delivery of cardiac therapy may be referred to as “baseline” electrical activity because no therapy is delivered to the patient such that the patient’s heart is in its natural, or intrinsic, rhythm.

Although monitoring electrical activity 204 is the first process noted in the method 201, it is to be understood that monitoring electrical activity 204 occurs, or takes place, throughout the method 201 during the delivery of pacing therapy to evaluate, or test, various different pacing configurations and parameters as will be described further herein.

The measuring and generating data 202 of method 201 further includes generating electrical heterogeneity information (EHI) 206 based on monitored, or measured, electrical activity using the plurality of external electrodes. The EHI may be described as information, or data, representative of at least one of mechanical cardiac functionality and electrical cardiac functionality. The EHI and other cardiac therapy information may be described in U.S. Pat. No. 9,474,457 entitled “Metrics of Electrical Dyssynchrony and Electrical Activation Patterns from Surface ECG Electrodes” and issued on Oct. 25, 2016, which is hereby incorporated by reference it its entirety.

Electrical heterogeneity information (e.g., data) may be defined as information indicative of at least one of mechanical synchrony or dyssynchrony of the heart and/or electrical synchrony or dyssynchrony of the heart. In other words, electrical heterogeneity information may represent a surrogate of actual mechanical and/or electrical functionality of a patient’s heart. In at least one embodiment, relative changes in electrical heterogeneity information (e.g., from baseline heterogeneity information generated from data monitored during intrinsic cardiac activation to cardiac therapy heterogeneity information generated from data monitored during a delivery of cardiac therapy, from a first set of heterogeneity information generated from data monitored during a first cardiac therapy configuration to a second set of heterogeneity information generated from data monitored during a first cardiac therapy configuration, etc.) may be used to determine a surrogate value representative of the changes in hemodynamic response (e.g., acute changes in LV pressure gradients). The left ventricular pressure may be typically monitored invasively with a pressure sensor located in the left ventricle of a patient’s heart. As such, the use of electrical heterogeneity information to determine a surrogate value representative of the left ventricular pressure may avoid invasive monitoring using a left ventricular pressure sensor.

In at least one embodiment, the electrical heterogeneity information may include a standard deviation of ventricular activation times measured using some or all of the external electrodes, e.g., of the electrode apparatus 110. Further, local, or regional, electrical heterogeneity information may include standard deviations and/or averages of activation times measured using electrodes located in certain anatomic areas of the torso. For example, external electrodes on the left side of the torso of a patient may be used to compute local, or regional, left electrical heterogeneity information.

The electrical heterogeneity information may be generated using one or more various systems and/or methods. For example, electrical heterogeneity information may be generated using an array, or a plurality, of surface electrodes and/or imaging systems as described in U.S. Pat. No. 9,510,763 entitled “Assessing Intra-Cardiac Activation Patterns and Electrical Dyssynchrony” and issued on Dec. 6, 2016, U.S. Pat. No. 8,972,228 entitled “Assessing Intra-Cardiac Activation Patterns” and issued on Mar. 3, 2014, and U.S. Pat. No. 8,180,428 B2 entitled “Methods and Systems for use in Selecting Cardiac Pacing Sites” and issued on May 15, 2012, each of which is incorporated herein by reference in its entirety.

Electrical heterogeneity information may include one or more metrics or indices. For example, one of the metrics, or indices, of electrical heterogeneity may be a standard deviation of activation times (SDAT) measured using some or all of the electrodes on the surface of the torso of a patient. In some examples, the SDAT may be calculated using the estimated cardiac activation times over the surface of a model heart.

Another metric, or index, of electrical heterogeneity may be a left standard deviation of surrogate electrical activation times (LVED) monitored by external electrodes located proximate the left side of a patient. Further, another metric, or index, of electrical heterogeneity may include an average of surrogate electrical activation times (LVAT) monitored by external electrodes located proximate the left side of a patient. The LVED and LVAT may be determined (e.g., calculated, computed, etc.) from electrical activity measured only by electrodes proximate the left side of the patient, which may be referred to as “left” electrodes. The left electrodes may be defined as any surface electrodes located proximate the left ventricle, which includes region to left of the patient’s sternum and spine. In one embodiment, the left electrodes may include all anterior electrodes on the left of the sternum and all posterior electrodes to the left of the spine. In another embodiment, the left electrodes may include all anterior electrodes on the left of the sternum and all posterior electrodes. In yet another embodiment, the left electrodes may be designated based on the contour of the left and right sides of the heart as determined using imaging apparatus (e.g., x-ray, fluoroscopy, etc.).

Another illustrative metric, or index, of dyssynchrony may be a range of activation times (RAT) that may be computed as the difference between the maximum and the minimum torso-surface or cardiac activation times, e.g., overall, or for a region. The RAT reflects the span of activation times while the SDAT gives an estimate of the dispersion of the activation times from a mean. The SDAT also provides an estimate of the heterogeneity of the activation times, because if activation times are spatially heterogeneous, the individual activation times will be further away from the mean activation time, indicating that one or more regions of heart have been delayed in activation. In some examples, the RAT may be calculated using the estimated cardiac activation times over the surface of a model heart.

Another illustrative metric, or index, of electrical heterogeneity information may include estimates of a percentage of surface electrodes located within a particular region of interest for the torso or heart whose associated activation times are greater than a certain percentile, such as, for example the 70th percentile, of measured QRS complex duration or the determined activation times for surface electrodes. The region of interest may, e.g., be a posterior, left anterior, and/or left-ventricular region. The illustrative metric, or index, may be referred to as a percentage of late activation (PLAT). The PLAT may be described as providing an estimate of percentage of the region of interest, e.g., posterior and left-anterior area associated with the left ventricular area of heart, which activates late. A large value for PLAT may imply delayed activation of a substantial portion of the region, e.g., the left ventricle, and the potential benefit of electrical resynchronization through CRT by pre-exciting the late region, e.g., of left ventricle. In other examples, the PLAT may be determined for other subsets of electrodes in other regions, such as a right anterior region to evaluate delayed activation in the right ventricle. Furthermore, in some examples, the PLAT may be calculated using the estimated cardiac activation times over the surface of a model heart for either the whole heart or for a particular region, e.g., left or right ventricle, of the heart.

In one or more embodiments, the electrical heterogeneity information may include indicators of favorable changes in global cardiac electrical activation such as, e.g., described in Sweeney et al., “Analysis of Ventricular Activation Using Surface Electrocardiography to Predict Left Ventricular Reverse Volumetric Remodeling During Cardiac Resynchronization Therapy,” Circulation, 2010 Feb 9, 121(5): 626-34 and/or Van Deursen, et al., “Vectorcardiography as a Tool for Easy Optimization of Cardiac Resynchronization Therapy in Canine LBBB Hearts,” Circulation Arrhythmia and Electrophysiology, 2012 Jun 1, 5(3): 544-52, each of which is incorporated herein by reference in its entirety. Heterogeneity information may also include measurements of improved cardiac mechanical function measured by imaging or other systems to track motion of implanted leads within the heart as, e.g., described in Ryu et al., “Simultaneous Electrical and Mechanical Mapping Using 3D Cardiac Mapping System: Novel Approach for Optimal Cardiac Resynchronization Therapy,” Journal of Cardiovascular Electrophysiology, 2010 Feb, 21(2): 219-22, Sperzel et al., “Intraoperative Characterization of Interventricular Mechanical Dyssynchrony Using Electroanatomic Mapping System—A Feasibility Study,” Journal of Interventional Cardiac Electrophysiology, 2012 Nov, 35(2): 189-96, and/or U.S. Pat. No. 8,145,306 A1 entitled “Method for Optimizing CRT Therapy” and issued on Mar. 27, 2012, each of which is incorporated herein by reference in its entirety.

Similar to the monitoring 204, it is to be understood that generating EHI 206 occurs, or takes place, throughout the method 201 based on electrical activity monitored during the delivery of pacing therapy to evaluate, or test, various different pacing configurations and parameters as will be described further herein.

The measuring and generating data 202 of method 201 further includes measuring the patient’s intrinsic AV delay 208, which may be utilized in the method 201 to provide one or more AV pacing delays for the pacing therapy to be delivered and evaluated (e.g., the AV pacing delay may be ratio, fraction, or percentage of the measured intrinsic AV delay). For example, using the measured intrinsic AV delay among other things, an initial AV pacing delay may be generated, which may be used to when delivering pacing therapy during the process of selection of a left ventricular pacing vector 210. In at least one embodiment, a plurality of AV pacing delays such as, e.g., 40%, 50%, 60%, 70%, 80%, 90%, etc. of the measured intrinsic AV delay may be utilized to deliver pacing therapy and the sensing functionality of pacing device (e.g., implantable medical device) may be used to evaluate each of the AV pacing delays to determine which of the AV pacing delays to utilize thereby resulting in the initial AV pacing delay. In other words, an initial AV pacing delay may be determined by first measuring the patient’s intrinsic AV delay and then delivering pacing therapy at a plurality of AV pacing delays less than the intrinsic AV delay to find, or determine, an initial AV pacing delay. In at least one embodiment, MEDTRONIC’s CARDIOSYNC may be utilized to determine the initial AV pacing delay. It is to be understood that measuring a patient’s intrinsic AV delay 208 may be performed by one or both of an implantable medical device such as described therein with respect to FIGS. 7-9 and the electrode apparatus 100 described herein with respect to FIGS. 1-3.

Next, the method 201 includes selecting a left ventricular pacing vector 210. Selecting a left ventricular pacing vector 210 includes delivering pacing therapy using the plurality of different left ventricular pacing vectors 212 and the initial AV pacing delay. Each different left ventricular pacing vector may use a combination of pacing electrodes different from the remaining different left ventricular pacing vectors. Further, each pacing vector may utilize two or more different pacing electrodes. For example, one pacing vector may utilize two pacing electrodes on near the distal end of left ventricular pacing lead while another pacing vector may utilize three pacing electrodes on near the distal end of left ventricular pacing lead.

Further, it is to be understood that each of the pacing electrodes may be coupled to one or more leads implanted in, or proximate to, the patient’s heart. Further, in at least one embodiment, the cardiac therapy may be delivered by a lead-less electrode. Illustrative cardiac therapy using an implantable electrode and lead may be further described herein with reference to FIGS. 7-9. Although the systems and devices of FIGS. 7-9 include three leads, it is to be understood that the illustrative systems, methods, and devices described herein may be used with any type of cardiac pacing systems including no leads, less than three leads, and more than three leads. As described herein, although the cardiac therapy delivery may be described as being invasive, the illustrative systems, methods, and devices may be described as being noninvasive because the illustrative systems, methods, and devices may only initiate the delivery of and configure the cardiac therapy, and the illustrative systems, methods, and devices may further use electrical signals that are monitored, or taken, from the patient noninvasively. Further, illustrative cardiac therapy may utilize a leaded or leadless implantable cardiac device that includes a tissue-piercing electrode implantable from the triangle of Koch region of the right atrium through the right atrial endocardium and central fibrous body to deliver cardiac therapy to or sense electrical activity of the left ventricle in the basal and/or septal region of the left ventricular myocardium of a patient’s heart as described in U.S. Pat. No. 11,058,880 entitled “VfA Cardiac Therapy for Tachycardia” and issued on Jul. 13, 2021, and U.S. Pat. App. Pub. No. 2020/0069949 entitled “Adaptive VfA Cardiac Therapy” and filed on Aug. 30, 2019, each of which is incorporated by reference herein in their entireties.

As described herein, the electrical activity of the patient may be monitored 204 using the plurality of external electrodes during the delivery of the pacing therapy using the plurality of different left ventricular pacing vectors 212. Further, EHI may be generated 206 based on such electrical activity monitored during the delivery of the pacing therapy using the plurality of different left ventricular pacing vectors 212. The EHI generated from the electrical activity monitored during the delivery of the pacing therapy using the plurality of different left ventricular pacing vectors 212 may then be used to determine which of the left ventricular pacing vectors should be selected 214. For example, the EHI generated from each different left ventricular pacing vector may be compared to one another or may be ranked, and the left ventricular pacing vector having the best EHI may be determined or selected (e.g., the best EHI may indicate the largest improvement in cardiac functionality).

For example, SDAT may be generated for each of the plurality of different left ventricular pacing vectors, and a left ventricular pacing vector of the plurality of different left ventricular pacing vectors may be selected based on the SDAT. For instance, the left ventricular pacing vector that provides the lowest, or smallest, SDAT may be determined or selected 214. Further, for example, a capture threshold may be generated for each of the plurality of different left ventricular pacing vectors, and a left ventricular pacing vector of the plurality of different left ventricular pacing vectors may be selected based on the capture thresholds. For instance, the left ventricular pacing vector that provides the lowest, or smallest, capture threshold may be determined or selected 214. Additionally, in one or more embodiments, any left ventricular pacing vectors that cause phrenic nerve stimulation may be excluded from selection.

As a result, the method 201 will have selected a left ventricular pacing vector 210 to be used in the pacing therapy. The selected left ventricular pacing vector may be utilized for pacing the left ventricle of the patient’s heart during the remainder of the processes, or tests, to configure the pacing therapy as well as the finally configured pacing therapy to be provided, or delivered, to the patient’s heart upon completion of method 201. Additionally, it is to be understood that the plurality of left ventricular pacing vectors may be configured (e.g., culled down, curated, limited, etc.) by a user or automatically prior to trying each of left ventricular pacing vector based on one or more factors such as, e.g., pacing capture threshold (PCT), phrenic nerve stimulation, less desirable lead and electrode locations, etc. Other illustrative systems and methods of determination or selection of pacing vector may be described in U.S. Pat. No. 10,064,567 entitled “Systems, Methods, and Interfaces for Identifying Optimal Electrical Vectors” and issued on Sep. 4, 2018, which is incorporated by reference in its entirety.

Next, the method 201 includes selecting one of adaptive pacing therapy or non-adaptive pacing therapy 220. Selecting one of adaptive pacing therapy or non-adaptive pacing therapy 220 may be generally described as testing adaptive pacing therapy using the selected left ventricular pacing vector to determine whether adaptive pacing therapy provides acceptable therapy. Acceptable therapy may be generally described as therapy that adequately treats a patient’s condition to a desired or intended level. In other words, acceptable therapy may be described as being effective therapy to treat a patient’s condition.

Selecting one of adaptive pacing therapy or non-adaptive pacing therapy 220 includes delivering adaptive pacing therapy 222 and analyzing electrical activity monitored using the plurality of external electrodes during the delivery of the adaptive pacing therapy to determine whether the adaptive pacing therapy is acceptable 224. If the adaptive pacing therapy is determined to be acceptable, the therapy configuration may be completed 299 and the remainder of the method 201 may not be pursued. For example, the illustrative systems and methods may provide an indication to one or more of a user (e.g., physician, a nurse, a patient, etc.), the pacing device (e.g., an IMD), and a programmer that the pacing configuration is completed. The indication may include, among other, things, the selected left ventricular pacing vector and the selection of adaptive pacing therapy. If the adaptive pacing therapy is not determined to be acceptable, the method 201 may continue to selection of VV pacing delay 230.

As described herein, the electrical activity of the patient may be measured, or monitored, 204 using the plurality of external electrodes during the delivery of the adaptive pacing therapy 222 using the selected left ventricular pacing vector. Further, EHI may be generated 206 based on such electrical activity measured, or monitored, during the delivery of the adaptive pacing therapy. The EHI generated from the electrical activity measured, or monitored, during the delivery of the adaptive pacing therapy using the selected left ventricular pacing vector may then be used to determine whether the adaptive pacing therapy is acceptable 224. For example, the EHI generated from during delivery of the adaptive pacing therapy may be compared to baseline EHI (which may be generated prior to delivery of any pacing therapy during intrinsic cardiac activation) or may be compared to a fixed threshold value.

For example, a SDAT may be generated for the adaptive pacing therapy, and the generated SDAT may be compared to an acceptable therapy threshold value. The acceptable therapy threshold value when using SDAT may be between about 10 milliseconds (ms) and 35 ms. In one embodiment, the acceptable therapy threshold value when using SDAT may be 20 ms. In one or more embodiments, the acceptable therapy threshold when using SDAT may be greater than or equal to 10 ms, greater than or equal to 15 ms, greater than or equal to 20 ms, etc. and/or less than or equal to 35 ms, less than or equal to 30 ms, less than or equal to 25 ms, etc. More specifically, the SDAT from the electrical activity measured, or monitored, during delivery of adaptive pacing therapy using the selected left ventricular pacing vector may be evaluated to determine whether it is less than or equal to an acceptable therapy threshold value to determine whether adaptive pacing therapy is acceptable. If the generated SDAT is less than or equal to the acceptable therapy threshold value, then the adaptive pacing therapy may be accepted and the therapy configuration may be completed 299, i.e., the final pacing therapy configuration is adaptive pacing therapy using the selected left ventricular pacing vector. As described herein, an indication, e.g., including the final pacing therapy configuration, may be provided by the illustrative systems and methods to one or more of a user, a pacing device, and programmer when the therapy configuration is completed. Additionally, it is to be understood that providing a pacing configuration to a pacing device may include instructing, or programming, the pacing device to behave, or perform, according to the provided pacing configuration. Additionally, it is to be understood that providing a pacing configuration to a pacing device may include providing the pacing configuration (e.g., including left ventricular pacing vector, atrioventricular pacing delay, interventricular pacing delay, adaptative or non-adaptive pacing configuration, etc.) to a pacing programmer, which then may be used to instruct, or program, the pacing device to behave, or perform, according to the provided pacing configuration.

Further, for example, SDAT may be generated for the adaptive pacing therapy, and the generated SDAT may be compared to a baseline SDAT that was generated from electrical activity monitored during intrinsic activation without delivery of pacing therapy. In this example, a percentage improvement between the SDAT generated during adaptive pacing therapy and the SDAT generated during intrinsic activation may be calculated and then compared to an acceptable therapy threshold percentage. For example, if percentage improvement between the SDAT generated during adaptive pacing therapy and the SDAT generated during intrinsic activation is greater than or equal to the acceptable therapy threshold percentage, then it may be determined that the adaptive pacing therapy is acceptable. The acceptable therapy threshold percentage when using SDAT may be between about 5% and 35 %. In one embodiment, the acceptable therapy threshold percentage when using SDAT may be 25%. In one or more embodiments, the acceptable therapy threshold percentage when using SDAT may be greater than or equal to 5%, greater than or equal to 10%, greater than or equal to 15%, greater than or equal to 20%, greater than or equal to 25%, etc. and/or less than or equal to 35%, less than or equal to 30 %, etc. More specifically, acceptable therapy threshold percentage may be described as the minimum percent improvement to indicate acceptable pacing therapy.

As described herein, if the adaptive pacing therapy is not determined to be acceptable, the method 201 may continue to selection of interventricular (VV) pacing delay 230 for use in non-adaptive cardiac therapy. The VV pacing delay 230 may be defined as the time period between delivery of a left ventricular pace and a right ventricular pace. Negative values of VV pacing delay indicate that the right ventricular pace is delivered prior to the left ventricular pace. Selecting a VV pacing delay 230 includes delivering pacing therapy using the selected left ventricular pacing vector and the initial AV pacing delay at a plurality of different VV pacing delays 232. For example, the plurality of different VV pacing delays may be between about -40 ms and 90 ms. For example, the plurality of different VV pacing delays may include -40 ms, -30 ms, -20 ms, -10 ms, 0 ms, 10 ms, 20 ms, 30 ms, 40 ms, 50 ms, 60 ms, 70 ms, 80 ms, etc. In one embodiment, six or less different VV pacing delays (e.g., -20 ms, 0 ms, 20 ms, 40 ms, 60 ms, 80 ms) are utilized. Additionally, -20 ms or 80 ms may not be used based on one or more conditions. For example, if the patient’s intrinsic LVAT is less than 28 ms, then the 80 VV pacing delay may not be used. Further, for example, if the patient’s intrinsic SDAT is less than 24 ms, then the -20 VV pacing delay may not be used.

As described herein, the electrical activity of the patient may be monitored 204 using the plurality of external electrodes during the delivery of the pacing therapy using the plurality of different VV pacing delays 232 using the selected left ventricular pacing vector. Further, EHI may be generated 206 based on such electrical activity monitored during the delivery of the pacing therapy using the plurality of different VV pacing delays 232 and the selected left ventricular pacing vector. The EHI generated from the electrical activity monitored during the delivery of the pacing therapy using the plurality of different VV pacing delays 232 may then be used to determine which of the VV pacing delays should be selected 234. For example, the EHI generated from each different VV pacing delay may be compared to one another or may be ranked, and the VV pacing delay having the best EHI may be determined or selected (e.g., the best EHI may indicate the largest improvement in cardiac functionality).

For example, a SDAT may be generated for each of the plurality of different VV pacing delays, and a VV pacing delay of the plurality of different VV pacing delays may be selected based on the SDAT. For instance, the VV pacing delay that provides the lowest, or smallest, SDAT may be determined or selected 234.

As a result, the method 201 will have selected a VV pacing delay 230 to be used in the pacing therapy. The selected VV pacing delay may then be utilized for pacing the left ventricle of the patient’s heart during the remainder of the processes, or tests, to configure the pacing therapy as well as the final pacing therapy to be provided, or delivered, to the patient’s heart upon completion of method 201. Other illustrative systems and methods of determination or selection of VV pacing delay may be described in U.S. Pat. No. 9,764,143 entitled “Systems and Methods for Configuration of Interventricular Interval” and issued on Sep. 19, 2017, which is incorporated by reference in its entirety.

The method 201 may then determine whether the pacing therapy is acceptable 236. More specifically, the method 201 may determine whether the non-adaptive pacing therapy using the selected left ventricular pacing vector and the selected VV delay is acceptable 236. The determination of whether the non-adaptive pacing therapy using the selected left ventricular pacing vector and the selected VV delay is acceptable 236 may utilize one or both of an acceptable therapy threshold value and an acceptable therapy threshold percentage in substantially the same or similar way as process 224 described herein. For example, The EHI, e.g., SDAT, for the electrical activity monitored during delivery of pacing therapy using the selected left ventricular pacing vector and the selected VV pacing delay may be compared to an acceptable therapy threshold value. If the EHI, e.g., SDAT, is less than or equal to the acceptable therapy threshold value, then the non-adaptive pacing therapy using the selected left ventricular pacing vector and selected VV pacing delay may be accepted and the therapy configuration may be completed 299, i.e., the final pacing therapy configuration is non-adaptive pacing therapy using the selected left ventricular pacing vector and the selected VV pacing delay. As described herein, an indication, e.g., including the final pacing therapy configuration, may be provided by the illustrative systems and methods to one or more of a user, a pacing device, and programmer when the therapy configuration is completed. Additionally, it is to be understood that providing a pacing configuration to a pacing device may include instructing, or programming, the pacing device to behave, or perform, according to the provided pacing configuration. Additionally, it is to be understood that providing a pacing configuration to a pacing device may include instructing, or programming, the pacing device to behave, or perform, according to the provided pacing configuration.

If the pacing therapy is not determined to be acceptable, the method 201 may continue to selection of AV pacing delay 240. An AV pacing delay may be defined as the delay between an intrinsic atrial sense or atrial pace and a left ventricular pace. Selecting an AV pacing delay 240 includes delivering pacing therapy using the selected left ventricular pacing vector and the selected VV pacing delay at a plurality of different AV pacing delays 242. The AV pacing delay may be defined in multiple ways. In one embodiment, the AV pacing delay may be a timing percentage of a patient’s intrinsic AV timing. For instance, the plurality of AV pacing delays can be between about 20% of a patient’s intrinsic AV delay and about 95% of a patient’s intrinsic AV delay. For example, the plurality of different AV pacing delays may include 20%, 30%, 35%, 40%, 45%, 50%, 55%, 60%, 65%, 70%, 75%, 80%, 90% etc. of a patient’s intrinsic AV delay. In at least one embodiment, only the following three AV pacing delays are utilized: 40%, 60 %, and 80% of a patient’s intrinsic AV delay.

As described herein, the electrical activity of the patient may be monitored 204 using the plurality of external electrodes during the delivery of the pacing therapy using the plurality of different AV pacing delays 242 using the selected left ventricular pacing vector and the selected VV pacing delay. Further, EHI may be generated 206 based on such electrical activity monitored during the delivery of the pacing therapy using the selected left ventricular pacing vector, the selected VV pacing delay, and the plurality of different AV pacing delays 242. The EHI generated from the electrical activity monitored during the delivery of the pacing therapy using the plurality of different AV pacing delays 242 may then be used to determine which of the AV pacing delays should be selected 244. For example, the EHI generated from each different AV pacing delay may be compared to one another or ranked, and the AV pacing delay having the best EHI may be determined or selected (e.g., the best EHI may indicate the largest improvement in cardiac functionality).

For example, a SDAT may be generated for each of the plurality of different AV pacing delays, and the AV pacing delay of the plurality of different AV pacing delays may be selected based on the SDAT. For instance, the AV pacing delay that provides the lowest, or smallest, SDAT may be determined or selected 244.

As a result, the method 201 will have selected an AV pacing delay 240 to be used in the pacing therapy. Other illustrative systems and methods of determination or selection of AV pacing delay may be described in U.S. Pat. No. 9,586,050 entitled “Systems and Methods for Configuration of Atrioventricular Interval” and issued on Mar. 7, 2017, which is incorporated by reference in its entirety.

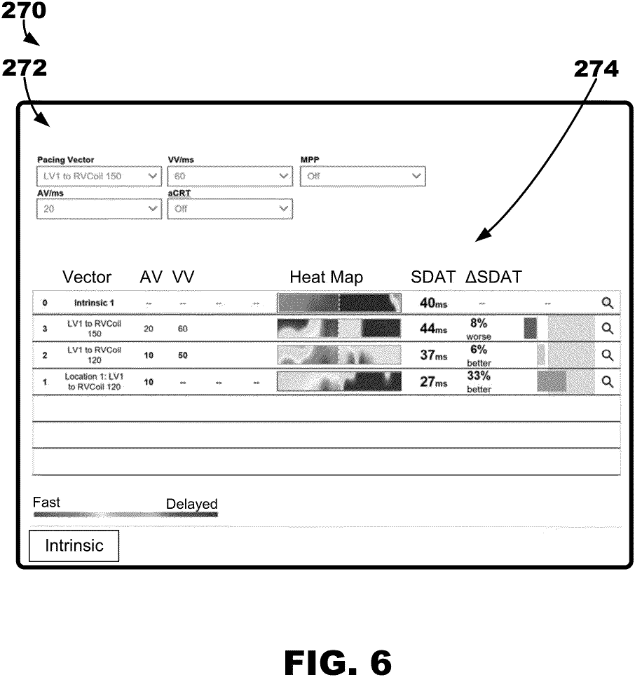

The selected ventricular pacing vector, the selected VV pacing delay, and the selected AV pacing delay may then be utilized for delivering pacing therapy, and such pacing therapy may then be evaluated to determine whether it is acceptable 246. More specifically, the method 201 may determine whether the non-adaptive pacing therapy using the selected left ventricular pacing vector, the selected VV delay, and the selected AV delay is acceptable 246. The determination of whether the non-adaptive pacing therapy using the selected left ventricular pacing vector, the selected VV delay, and the selected AV delay is acceptable 246 may utilize one or both of an acceptable therapy threshold value and an acceptable therapy threshold percentage in substantially the same or similar way as process 224 described herein. For example, the EHI, e.g., SDAT, for the electrical activity monitored during delivery of pacing therapy using the selected left ventricular pacing vector, the selected VV delay, and the selected AV delay may be compared to an acceptable therapy threshold value. If the EHI, e.g., SDAT, is less than or equal to the acceptable therapy threshold value, then the non-adaptive pacing therapy using the selected left ventricular pacing vector, the selected VV delay, and the selected AV delay may be accepted and the therapy configuration may be completed 299, i.e., the final pacing therapy configuration is non-adaptive pacing therapy using the selected left ventricular pacing vector, the selected VV delay, and the selected AV delay. If the pacing therapy is not determined to be acceptable, the method 201 may provide an indication to a user that the final configuration was not acceptable 250 using, for example, the illustrative graphical user interface depicted in FIG. 6.

Although as described herein with respect to FIGS. 5A-5B, the pacing therapy is evaluated for acceptability at three different locations or times within the method 201, it is to be understood that the pacing therapy acceptability evaluation may occur continuously throughout method 201. Thus, at any time within method 201, the method 201 may determine that the current pacing configuration is acceptable, and thus, determine that the pacing therapy configuration is complete thereby halting the remainder of the method 201. In this way, if the method 201 finds an acceptable pacing configuration, the method 201 can stop without spending any more time searching other pacing settings or configurations.

An illustrative graphical user interface (GUI) 270 to be used with the methods of evaluation and configuration of cardiac therapy of FIGS. 4-5. As shown, the GUI 270 includes a selection region 272 where a user may select the pacing vector, VV pacing delay, AV pacing delay, adaptive or non-adaptive pacing therapy, and multipoint pacing, which may program a pacing device to deliver pacing using such selected configuration. The GUI 270 further includes a list region 274 including a plurality of different pacing configurations and the metrics, e.g., SDAT and SDAT change from intrinsic activation, resulting from each respective configuration. As shown, the pacing therapy configuration 1 utilizing Location 1: LV1 to RVCoil 120, an AV pacing delay of 10 ms, and no VV pacing delay resulted in an SDAT of 27 ms, which was 33% better than intrinsic.

Illustrative cardiac therapy systems and devices may be further described herein with reference to FIGS. 7-9 that may utilizes the illustrative systems, interfaces, methods, and processes described herein with respect to FIGS. 1-6.

FIG. 7 is a conceptual diagram illustrating an illustrative therapy system 10 that may be used to deliver pacing therapy to a patient 14. Patient 14 may, but not necessarily, be a human. The therapy system 10 may include an implantable medical device 16 (IMD), which may be coupled to leads 18, 20, 22. The IMD 16 may be, e.g., an implantable pacemaker, cardioverter, and/or defibrillator, that delivers, or provides, electrical signals (e.g., paces, etc.) to and/or senses electrical signals from the heart 12 of the patient 14 via electrodes coupled to one or more of the leads 18, 20, 22.

The leads 18, 20, 22 extend into the heart 12 of the patient 14 to sense electrical activity of the heart 12 and/or to deliver electrical stimulation to the heart 12. In the example shown in FIG. 7, the right ventricular (RV) lead 18 extends through one or more veins (not shown), the superior vena cava (not shown), and the right atrium 26, and into the right ventricle 28. The left ventricular (LV) coronary sinus lead 20 extends through one or more veins, the vena cava, the right atrium 26, and into the coronary sinus 30 to a region adjacent to the free wall of the left ventricle 32 of the heart 12. The right atrial (RA) lead 22 extends through one or more veins and the vena cava, and into the right atrium 26 of the heart 12.

The IMD 16 may sense, among other things, electrical signals attendant to the depolarization and repolarization of the heart 12 via electrodes coupled to at least one of the leads 18, 20, 22. In some examples, the IMD 16 provides pacing therapy (e.g., pacing pulses) to the heart 12 based on the electrical signals sensed within the heart 12. The IMD 16 may be operable to adjust one or more parameters associated with the pacing therapy such as, e.g., A-V delay and other various timings, pulse wide, amplitude, voltage, burst length, etc. Further, the IMD 16 may be operable to use various electrode configurations to deliver pacing therapy, which may be unipolar, bipolar, quadripoloar, or further multipolar. For example, a multipolar lead may include several electrodes that can be used for delivering pacing therapy. Hence, a multipolar lead system may provide, or offer, multiple electrical vectors to pace from. A pacing vector may include at least one cathode, which may be at least one electrode located on at least one lead, and at least one anode, which may be at least one electrode located on at least one lead (e.g., the same lead, or a different lead) and/or on the casing, or can, of the IMD. While improvement in cardiac function as a result of the pacing therapy may primarily depend on the cathode, the electrical parameters like impedance, pacing threshold voltage, current drain, longevity, etc. may be more dependent on the pacing vector, which includes both the cathode and the anode. The IMD 16 may also provide defibrillation therapy and/or cardioversion therapy via electrodes located on at least one of the leads 18, 20, 22. Further, the IMD 16 may detect arrhythmia of the heart 12, such as fibrillation of the ventricles 28, 32, and deliver defibrillation therapy to the heart 12 in the form of electrical pulses. In some examples, IMD 16 may be programmed to deliver a progression of therapies, e.g., pulses with increasing energy levels, until a fibrillation of heart 12 is stopped.

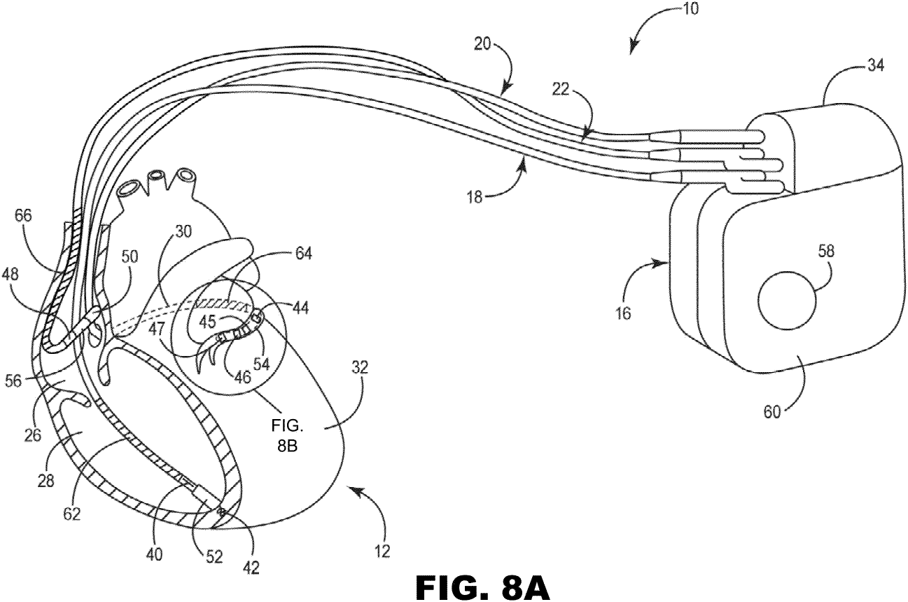

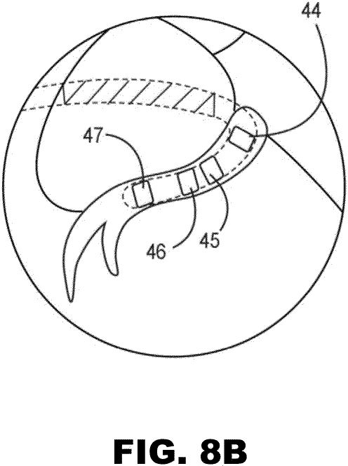

FIGS. 8A-8B are conceptual diagrams illustrating the IMD 16 and the leads 18, 20, 22 of therapy system 10 of FIG. 7 in more detail. The leads 18, 20, 22 may be electrically coupled to a therapy delivery module (e.g., for delivery of pacing therapy), a sensing module (e.g., for sensing one or more signals from one or more electrodes), and/or any other modules of the IMD 16 via a connector block 34. In some examples, the proximal ends of the leads 18, 20, 22 may include electrical contacts that electrically couple to respective electrical contacts within the connector block 34 of the IMD 16. In addition, in some examples, the leads 18, 20, 22 may be mechanically coupled to the connector block 34 with the aid of set screws, connection pins, or another suitable mechanical coupling mechanism.

Each of the leads 18, 20, 22 includes an elongated insulative lead body, which may carry a number of conductors (e.g., concentric coiled conductors, straight conductors, etc.) separated from one another by insulation (e.g., tubular insulative sheaths). In the illustrated example, bipolar electrodes 40, 42 are located proximate to a distal end of the lead 18. In addition, bipolar electrodes 44, 45, 46, 47 are located proximate to a distal end of the lead 20 and bipolar electrodes 48, 50 are located proximate to a distal end of the lead 22.

The electrodes 40, 44, 45, 46, 47, 48 may take the form of ring electrodes, and the electrodes 42, 50 may take the form of extendable helix tip electrodes mounted retractably within the insulative electrode heads 52, 54, 56, respectively. Each of the electrodes 40, 42, 44, 45, 46, 47, 48, 50 may be electrically coupled to a respective one of the conductors (e.g., coiled and/or straight) within the lead body of its associated lead 18, 20, 22, and thereby coupled to a respective one of the electrical contacts on the proximal end of the leads 18, 20, 22.

Additionally, electrodes 44, 45, 46 and 47 may have an electrode surface area of about 5.3 mm² to about 5.8 mm². Electrodes 44, 45, 46, and 47 may also be referred to as LV1, LV2, LV3, and LV4, respectively. The LV electrodes (i.e., left ventricle electrode 1 (LV1) 44, left ventricle electrode 2 (LV2) 45, left ventricle electrode 3 (LV3) 46, and left ventricle 4 (LV4) 47 etc.) on the lead 20 can be spaced apart at variable distances. For example, electrode 44 may be a distance of, e.g., about 21 millimeters (mm), away from electrode 45, electrodes 45 and 46 may be spaced a distance of, e.g. about 1.3 mm to about 1.5 mm, away from each other, and electrodes 46 and 47 may be spaced a distance of, e.g. 20 mm to about 21 mm, away from each other.

The electrodes 40, 42, 44, 45, 46, 47, 48, 50 may further be used to sense electrical signals (e.g., morphological waveforms within electrograms (EGM)) attendant to the depolarization and repolarization of the heart 12. The electrical signals are conducted to the IMD 16 via the respective leads 18, 20, 22. In some examples, the IMD 16 may also deliver pacing pulses via the electrodes 40, 42, 44, 45, 46, 47, 48, 50 to cause depolarization of cardiac tissue of the patient’s heart 12. In some examples, as illustrated in FIG. 8A, the IMD 16 includes one or more housing electrodes, such as housing electrode 58, which may be formed integrally with an outer surface of a housing 60 (e.g., hermetically-sealed housing) of the IMD 16 or otherwise coupled to the housing 60. Any of the electrodes 40, 42, 44, 45, 46, 47, 48, 50 may be used for unipolar sensing or pacing in combination with the housing electrode 58. It is generally understood by those skilled in the art that other electrodes can also be selected to define, or be used for, pacing and sensing vectors. Further, any of electrodes 40, 42, 44, 45, 46, 47, 48, 50, 58, when not being used to deliver pacing therapy, may be used to sense electrical activity during pacing therapy.

As described in further detail with reference to FIG. 8A, the housing 60 may enclose a therapy delivery module that may include a stimulation generator for generating cardiac pacing pulses and defibrillation or cardioversion shocks, as well as a sensing module for monitoring the electrical signals of the patient’s heart (e.g., the patient’s heart rhythm). The leads 18, 20, 22 may also include elongated electrodes 62, 64, 66, respectively, which may take the form of a coil. The IMD 16 may deliver defibrillation shocks to the heart 12 via any combination of the elongated electrodes 62, 64, 66 and the housing electrode 58. The electrodes 58, 62, 64, 66 may also be used to deliver cardioversion pulses to the heart 12. Further, the electrodes 62, 64, 66 may be fabricated from any suitable electrically conductive material, such as, but not limited to, platinum, platinum alloy, and/or other materials known to be usable in implantable defibrillation electrodes. Since electrodes 62, 64, 66 are not generally configured to deliver pacing therapy, any of electrodes 62, 64, 66 may be used to sense electrical activity and may be used in combination with any of electrodes 40, 42, 44, 45, 46, 47, 48, 50, 58. In at least one embodiment, the RV elongated electrode 62 may be used to sense electrical activity of a patient’s heart during the delivery of pacing therapy (e.g., in combination with the housing electrode 58, or defibrillation electrode-to-housing electrode vector).

The configuration of the illustrative therapy system 10 illustrated in FIGS. 7-9 is merely one example. In other examples, the therapy system may include epicardial leads and/or patch electrodes instead of or in addition to the transvenous leads 18, 20, 22 illustrated in FIG. 7. Additionally, in other examples, the therapy system 10 may be implanted in/around the cardiac space without transvenous leads (e.g., leadless/wireless pacing systems) or with leads implanted (e.g., implanted transvenously or using approaches) into the left chambers of the heart (in addition to or replacing the transvenous leads placed into the right chambers of the heart as illustrated in FIG. 7). Further, in one or more embodiments, the IMD 16 need not be implanted within the patient 14. For example, the IMD 16 may deliver various cardiac therapies to the heart 12 via percutaneous leads that extend through the skin of the patient 14 to a variety of positions within or outside of the heart 12. In one or more embodiments, the system 10 may utilize wireless pacing (e.g., using energy transmission to the intracardiac pacing component(s) via ultrasound, inductive coupling, RF, etc.) and sensing cardiac activation using electrodes on the can/housing and/or on subcutaneous leads.

In other examples of therapy systems that provide electrical stimulation therapy to the heart 12, such therapy systems may include any suitable number of leads coupled to the IMD 16, and each of the leads may extend to any location within or proximate to the heart 12. For example, other examples of therapy systems may include three transvenous leads located as illustrated in FIGS. 7-9. Still further, other therapy systems may include a single lead that extends from the IMD 16 into the right atrium 26 or the right ventricle 28, or two leads that extend into a respective one of the right atrium 26 and the right ventricle 28.

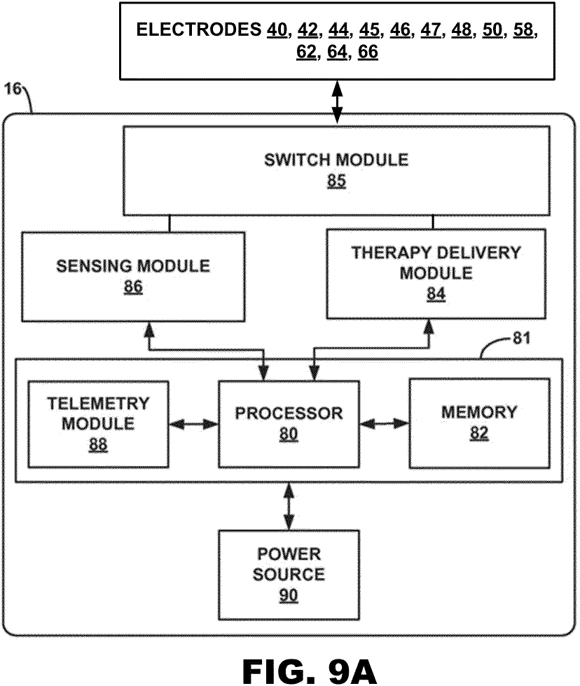

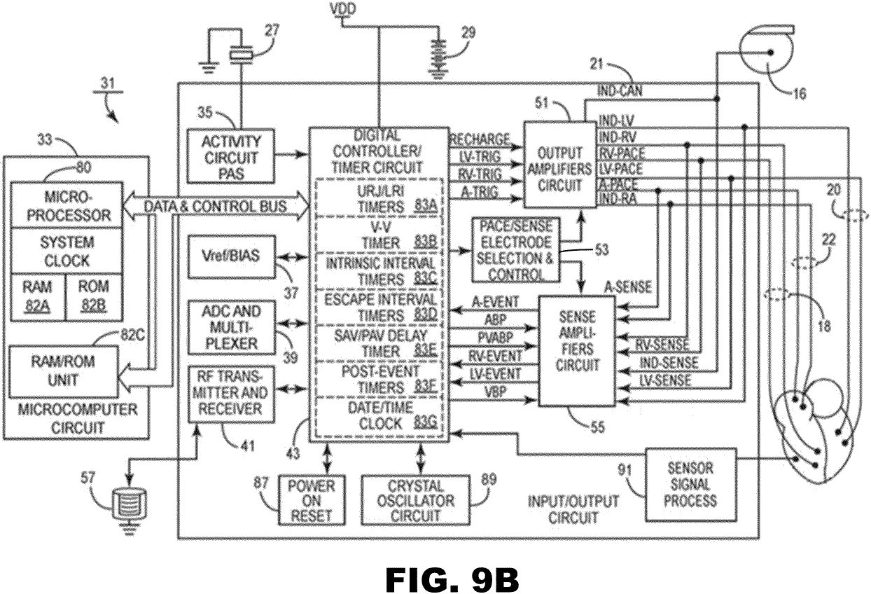

FIG. 9A is a functional block diagram of one illustrative configuration of the IMD 16. As shown, the IMD 16 may include a control module 81, a therapy delivery module 84 (e.g., which may include a stimulation generator), a sensing module 86, and a power source 90.

The control module, or apparatus, 81 may include a processor 80, memory 82, and a telemetry module, or apparatus, 88. The memory 82 may include computer-readable instructions that, when executed, e.g., by the processor 80, cause the IMD 16 and/or the control module 81 to perform various functions attributed to the IMD 16 and/or the control module 81 described herein. Further, the memory 82 may include any volatile, non-volatile, magnetic, optical, and/or electrical media, such as a random-access memory (RAM), read-only memory (ROM), non-volatile RAM (NVRAM), electrically-erasable programmable ROM (EEPROM), flash memory, and/or any other digital media. An illustrative capture management module may be the left ventricular capture management (LVCM) module described in U.S. Pat. No. 7,684,863 entitled “LV THRESHOLD MEASUREMENT AND CAPTURE MANAGEMENT” and issued Mar. 23, 2010, which is incorporated herein by reference in its entirety.

The processor 80 of the control module 81 may include any one or more of a microprocessor, a controller, a digital signal processor (DSP), an application specific integrated circuit (ASIC), a field-programmable gate array (FPGA), and/or equivalent discrete or integrated logic circuitry. In some examples, the processor 80 may include multiple components, such as any combination of one or more microprocessors, one or more controllers, one or more DSPs, one or more ASICs, and/or one or more FPGAs, as well as other discrete or integrated logic circuitry. The functions attributed to the processor 80 herein may be embodied as software, firmware, hardware, or any combination thereof.

The control module 81 may control the therapy delivery module 84 to deliver therapy (e.g., electrical stimulation therapy such as pacing) to the heart 12 according to a selected one or more therapy programs, which may be stored in the memory 82. More, specifically, the control module 81 (e.g., the processor 80) may control various parameters of the electrical stimulus delivered by the therapy delivery module 84 such as, e.g., A-V delays, V-V delays, pacing pulses with the amplitudes, pulse widths, frequency, or electrode polarities, etc., which may be specified by one or more selected therapy programs (e.g., A-V and/or V-V delay adjustment programs, pacing therapy programs, pacing recovery programs, capture management programs, etc.). As shown, the therapy delivery module 84 is electrically coupled to electrodes 40, 42, 44, 45, 46, 47, 48, 50, 58, 62, 64, 66, e.g., via conductors of the respective lead 18, 20, 22, or, in the case of housing electrode 58, via an electrical conductor disposed within housing 60 of IMD 16. Therapy delivery module 84 may be configured to generate and deliver electrical stimulation therapy such as pacing therapy to the heart 12 using one or more of the electrodes 40, 42, 44, 45, 46, 47, 48, 50, 58, 62, 64, 66.

For example, therapy delivery module 84 may deliver pacing stimulus (e.g., pacing pulses) via ring electrodes 40, 44, 45, 46, 47, 48 coupled to leads 18, 20, 22 and/or helical tip electrodes 42, 50 of leads 18, 22. Further, for example, therapy delivery module 84 may deliver defibrillation shocks to heart 12 via at least two of electrodes 58, 62, 64, 66. In some examples, therapy delivery module 84 may be configured to deliver pacing, cardioversion, or defibrillation stimulation in the form of electrical pulses. In other examples, therapy delivery module 84 may be configured deliver one or more of these types of stimulation in the form of other signals, such as sine waves, square waves, and/or other substantially continuous time signals.

The IMD 16 may further include a switch module 85 and the control module 81 (e.g., the processor 80) may use the switch module 85 to select, e.g., via a data/address bus, which of the available electrodes are used to deliver therapy such as pacing pulses for pacing therapy, or which of the available electrodes are used for sensing. The switch module 85 may include a switch array, switch matrix, multiplexer, or any other type of switching device suitable to selectively couple the sensing module 86 and/or the therapy delivery module 84 to one or more selected electrodes. More specifically, the therapy delivery module 84 may include a plurality of pacing output circuits. Each pacing output circuit of the plurality of pacing output circuits may be selectively coupled, e.g., using the switch module 85, to one or more of the electrodes 40, 42, 44, 45, 46, 47, 48, 50, 58, 62, 64, 66 (e.g., a pair of electrodes for delivery of therapy to a bipolar or multipolar pacing vector). In other words, each electrode can be selectively coupled to one of the pacing output circuits of the therapy delivery module using the switching module 85.

The sensing module 86 is coupled (e.g., electrically coupled) to sensing apparatus, which may include, among additional sensing apparatus, the electrodes 40, 42, 44, 45, 46, 47, 48, 50, 58, 62, 64, 66 to monitor electrical activity of the heart 12, e.g., electrocardiogram (ECG)/electrogram (EGM) signals, etc. The ECG/EGM signals may be used to measure or monitor activation times (e.g., ventricular activations times, etc.), heart rate (HR), heart rate variability (HRV), heart rate turbulence (HRT), deceleration/acceleration capacity, deceleration sequence incidence, T-wave altemans (TWA), P-wave to P-wave intervals (also referred to as the P-P intervals or A-A intervals), R-wave to R-wave intervals (also referred to as the R-R intervals or V-V intervals), P-wave to QRS complex intervals (also referred to as the P-R intervals, A-V intervals, or P-Q intervals), QRS-complex morphology, ST segment (i.e., the segment that connects the QRS complex and the T-wave), T-wave changes, QT intervals, electrical vectors, etc.

The switch module 85 may also be used with the sensing module 86 to select which of the available electrodes are used, or enabled, to, e.g., sense electrical activity of the patient’s heart (e.g., one or more electrical vectors of the patient’s heart using any combination of the electrodes 40, 42, 44, 45, 46, 47, 48, 50, 58, 62, 64, 66). Likewise, the switch module 85 may also be used with the sensing module 86 to select which of the available electrodes are not to be used (e.g., disabled) to, e.g., sense electrical activity of the patient’s heart (e.g., one or more electrical vectors of the patient’s heart using any combination of the electrodes 40, 42, 44, 45, 46, 47, 48, 50, 58, 62, 64, 66), etc. In some examples, the control module 81 may select the electrodes that function as sensing electrodes via the switch module within the sensing module 86, e.g., by providing signals via a data/address bus.

In some examples, sensing module 86 includes a channel that includes an amplifier with a relatively wider pass band than the R-wave or P-wave amplifiers. Signals from the selected sensing electrodes may be provided to a multiplexer, and thereafter converted to multi-bit digital signals by an analog-to-digital converter for storage in memory 82, e.g., as an electrogram (EGM). In some examples, the storage of such EGMs in memory 82 may be under the control of a direct memory access circuit.

In some examples, the control module 81 may operate as an interrupt-driven device and may be responsive to interrupts from pacer timing and control module, where the interrupts may correspond to the occurrences of sensed P-waves and R-waves and the generation of cardiac pacing pulses. Any necessary mathematical calculations may be performed by the processor 80 and any updating of the values or intervals controlled by the pacer timing and control module may take place following such interrupts. A portion of memory 82 may be configured as a plurality of recirculating buffers, capable of holding one or more series of measured intervals, which may be analyzed by, e.g., the processor 80 in response to the occurrence of a pace or sense interrupt to determine whether the patient’s heart 12 is presently exhibiting atrial or ventricular tachyarrhythmia.

The telemetry module 88 of the control module 81 may include any suitable hardware, firmware, software, or any combination thereof for communicating with another device, such as a programmer. For example, under the control of the processor 80, the telemetry module 88 may receive downlink telemetry from and send uplink telemetry to a programmer with the aid of an antenna, which may be internal and/or external. The processor 80 may provide the data to be uplinked to a programmer and the control signals for the telemetry circuit within the telemetry module 88, e.g., via an address/data bus. In some examples, the telemetry module 88 may provide received data to the processor 80 via a multiplexer.

The various components of the IMD 16 are further coupled to a power source 90, which may include a rechargeable or non-rechargeable battery. A non-rechargeable battery may be selected to last for several years, while a rechargeable battery may be inductively charged from an external device, e.g., on a daily or weekly basis.

FIG. 9B is another embodiment of a functional block diagram for IMD 16 that depicts bipolar RA lead 22, bipolar RV lead 18, and bipolar LV CS lead 20 without the LA CS pace/sense electrodes and coupled with an implantable pulse generator (IPG) circuit 31 having programmable modes and parameters of a bi-ventricular DDD/R type known in the pacing art. In turn, the sensor signal processing circuit 91 indirectly couples to the timing circuit 43 and via data and control bus to microcomputer circuitry 33. The IPG circuit 31 is illustrated in a functional block diagram divided generally into a microcomputer circuit 33 and a pacing circuit 21. The pacing circuit 21 includes the digital controller/timer circuit 43, the output amplifiers circuit 51, the sense amplifiers circuit 55, the RF telemetry transceiver 41, the activity sensor circuit 35 as well as a number of other circuits and components described below.

Crystal oscillator circuit 89 provides the basic timing clock for the pacing circuit 21 while battery 29 provides power. Power-on-reset circuit 87 responds to initial connection of the circuit to the battery for defining an initial operating condition and similarly, resets the operative state of the device in response to detection of a low battery condition. Reference mode circuit 37 generates stable voltage reference and currents for the analog circuits within the pacing circuit 21. Analog-to-digital converter (ADC) and multiplexer circuit 39 digitize analog signals and voltage to provide, e.g., real time telemetry of cardiac signals from sense amplifiers 55 for uplink transmission via RF transmitter and receiver circuit 41. Voltage reference and bias circuit 37, ADC and multiplexer 39, power-on-reset circuit 87, and crystal oscillator circuit 89 may correspond to any of those used in illustrative implantable cardiac pacemakers.

If the IPG is programmed to a rate responsive mode, the signals output by one or more physiologic sensors are employed as a rate control parameter (RCP) to derive a physiologic escape interval. For example, the escape interval is adjusted proportionally to the patient’s activity level developed in the patient activity sensor (PAS) circuit 35 in the depicted, illustrative IPG circuit 31. The patient activity sensor 27 is coupled to the IPG housing and may take the form of a piezoelectric crystal transducer. The output signal of the patient activity sensor 27 may be processed and used as an RCP. Sensor 27 generates electrical signals in response to sensed physical activity that are processed by activity circuit 35 and provided to digital controller/timer circuit 43. Activity circuit 35 and associated sensor 27 may correspond to the circuitry disclosed in U.S. Pat. No. 5,052,388 entitled “METHOD AND APPARATUS FOR IMPLEMENTING ACTIVITY SENSING IN A PULSE GENERATOR” and issued on Oct. 1, 1991 and U.S. Pat. No. 4,428,378 entitled “RATE ADAPTIVE PACER” and issued on Jan. 31, 1984, each of which is incorporated herein by reference in its entirety. Similarly, the illustrative systems, apparatus, and methods described herein may be practiced in conjunction with alternate types of sensors such as oxygenation sensors, pressure sensors, pH sensors, and respiration sensors, for use in providing rate responsive pacing capabilities. Alternately, QT time may be used as a rate indicating parameter, in which case no extra sensor is required. Similarly, the illustrative embodiments described herein may also be practiced in non-rate responsive pacemakers.

Data transmission to and from the external programmer is accomplished by way of the telemetry antenna 57 and an associated RF transceiver 41, which serves both to demodulate received downlink telemetry and to transmit uplink telemetry. Uplink telemetry capabilities may include the ability to transmit stored digital information, e.g., operating modes and parameters, EGM histograms, and other events, as well as real time EGMs of atrial and/or ventricular electrical activity and marker channel pulses indicating the occurrence of sensed and paced depolarizations in the atrium and ventricle.

Microcomputer 33 contains a microprocessor 80 and associated system clock and on-processor RAM and ROM chips 82A and 82B, respectively. In addition, microcomputer circuit 33 includes a separate RAM/ROM chip 82C to provide additional memory capacity. Microprocessor 80 normally operates in a reduced power consumption mode and is interrupt driven. Microprocessor 80 is awakened in response to defined interrupt events, which may include A-TRIG, RV-TRIG, LV-TRIG signals generated by timers in digital timer/controller circuit 43 and A-EVENT, RV-EVENT, and LV-EVENT signals generated by sense amplifiers circuit 55, among others. The specific values of the intervals and delays timed out by digital controller/timer circuit 43 are controlled by the microcomputer circuit 33 by way of data and control bus from programmed-in parameter values and operating modes. In addition, if programmed to operate as a rate responsive pacemaker, a timed interrupt, e.g., every cycle or every two seconds, may be provided in order to allow the microprocessor to analyze the activity sensor data and update the basic A-A, V-A, or V-V escape interval, as applicable. In addition, the microprocessor 80 may also serve to define variable, operative A-V delay intervals, V-V delay intervals, and the energy delivered to each ventricle and/or atrium.

In one embodiment, microprocessor 80 is a custom microprocessor adapted to fetch and execute instructions stored in RAM/ROM unit 82 in a conventional manner. It is contemplated, however, that other implementations may be suitable to practice the present disclosure. For example, an off-the-shelf, commercially available microprocessor or microcontroller, or custom application-specific, hardwired logic, or state-machine type circuit may perform the functions of microprocessor 80.

Digital controller/timer circuit 43 operates under the general control of the microcomputer 33 to control timing and other functions within the pacing circuit 21 and includes a set of timing and associated logic circuits of which certain ones pertinent to the present disclosure are depicted. The depicted timing circuits include URI/LRI timers 83A, V-V delay timer 83B, intrinsic interval timers 83C for timing elapsed V-EVENT to V-EVENT intervals or V-EVENT to A-EVENT intervals or the V-V conduction interval, escape interval timers 83D for timing A-A, V-A, and/or V-V pacing escape intervals, an A-V delay interval timer 83E for timing the A-LVp delay (or A-RVp delay) from a preceding A-EVENT or A-TRIG, a post-ventricular timer 83F for timing post-ventricular time periods, and a date/time clock 83G.

The A-V delay interval timer 83E is loaded with an appropriate delay interval for one ventricular chamber (e.g., either an A-RVp delay or an A-LVp) to time-out starting from a preceding A-PACE or A-EVENT. The interval timer 83E triggers pacing stimulus delivery and can be based on one or more prior cardiac cycles (or from a data set empirically derived for a given patient).

The post-event timer 83F times out the post-ventricular time period following an RV-EVENT or LV-EVENT or a RV-TRIG or LV-TRIG and post-atrial time periods following an A-EVENT or A-TRIG. The durations of the post-event time periods may also be selected as programmable parameters stored in the microcomputer 33. The post-ventricular time periods include the PVARP, a post-atrial ventricular blanking period (PAVBP), a ventricular blanking period (VBP), a post-ventricular atrial blanking period (PVARP) and a ventricular refractory period (VRP) although other periods can be suitably defined depending, at least in part, on the operative circuitry employed in the pacing engine. The post-atrial time periods include an atrial refractory period (ARP) during which an A-EVENT is ignored for the purpose of resetting any A-V delay, and an atrial blanking period (ABP) during which atrial sensing is disabled. It should be noted that the starting of the post-atrial time periods and the A-V delays can be commenced substantially simultaneously with the start or end of each A-EVENT or A-TRIG or, in the latter case, upon the end of the A-PACE which may follow the A-TRIG. Similarly, the starting of the post-ventricular time periods and the V-A escape interval can be commenced substantially simultaneously with the start or end of the V-EVENT or V-TRIG or, in the latter case, upon the end of the V-PACE which may follow the V-TRIG. The microprocessor 80 also optionally calculates A-V delays, V-V delays, post-ventricular time periods, and post-atrial time periods that vary with the sensor-based escape interval established in response to the RCP(s) and/or with the intrinsic atrial and/or ventricular rate.

The output amplifiers circuit 51 contains a RA pace pulse generator (and a LA pace pulse generator if LA pacing is provided), a RV pace pulse generator, a LV pace pulse generator, and/or any other pulse generator configured to provide atrial and ventricular pacing. In order to trigger generation of an RV-PACE or LV-PACE pulse, digital controller/timer circuit 43 generates the RV-TRIG signal at the time-out of the A-RVp delay (in the case of RV pre-excitation) or the LV-TRIG at the time-out of the A-LVp delay (in the case of LV pre-excitation) provided by A-V delay interval timer 83E (or the V-V delay timer 83B). Similarly, digital controller/timer circuit 43 generates an RA-TRIG signal that triggers output of an RA-PACE pulse (or an LA-TRIG signal that triggers output of an LA-PACE pulse, if provided) at the end of the V-A escape interval timed by escape interval timers 83D.

The output amplifiers circuit 51 includes switching circuits for coupling selected pace electrode pairs from among the lead conductors and the IND-CAN electrode to the RA pace pulse generator (and LA pace pulse generator if provided), RV pace pulse generator and LV pace pulse generator. Pace/sense electrode pair selection and control circuit 53 selects lead conductors and associated pace electrode pairs to be coupled with the atrial and ventricular output amplifiers within output amplifiers circuit 51 for accomplishing RA, LA, RV and LV pacing.

The sense amplifiers circuit 55 contains sense amplifiers for atrial and ventricular pacing and sensing. High impedance P-wave and R-wave sense amplifiers may be used to amplify a voltage difference signal that is generated across the sense electrode pairs by the passage of cardiac depolarization wavefronts. The high impedance sense amplifiers use high gain to amplify the low amplitude signals and rely on pass band filters, time domain filtering and amplitude threshold comparison to discriminate a P-wave or R-wave from background electrical noise. Digital controller/timer circuit 43 controls sensitivity settings of the atrial and ventricular sense amplifiers 55.

The sense amplifiers may be uncoupled from the sense electrodes during the blanking periods before, during, and after delivery of a pace pulse to any of the pace electrodes of the pacing system to avoid saturation of the sense amplifiers. The sense amplifiers circuit 55 includes blanking circuits for uncoupling the selected pairs of the lead conductors and the IND-CAN electrode from the inputs of the RA sense amplifier (and LA sense amplifier if provided), RV sense amplifier and LV sense amplifier during the ABP, PVABP and VBP. The sense amplifiers circuit 55 also includes switching circuits for coupling selected sense electrode lead conductors and the IND-CAN electrode to the RA sense amplifier (and LA sense amplifier if provided), RV sense amplifier and LV sense amplifier. Again, sense electrode selection and control circuit 53 selects conductors and associated sense electrode pairs to be coupled with the atrial and ventricular sense amplifiers within the output amplifiers circuit 51 and sense amplifiers circuit 55 for accomplishing RA, LA, RV, and LV sensing along desired unipolar and bipolar sensing vectors.

Right atrial depolarizations or P-waves in the RA-SENSE signal that are sensed by the RA sense amplifier result in a RA-EVENT signal that is communicated to the digital controller/timer circuit 43. Similarly, left atrial depolarizations or P-waves in the LA-SENSE signal that are sensed by the LA sense amplifier, if provided, result in a LA-EVENT signal that is communicated to the digital controller/timer circuit 43. Ventricular depolarizations or R-waves in the RV-SENSE signal are sensed by a ventricular sense amplifier result in an RV-EVENT signal that is communicated to the digital controller/timer circuit 43. Similarly, ventricular depolarizations or R-waves in the LV-SENSE signal are sensed by a ventricular sense amplifier result in an LV-EVENT signal that is communicated to the digital controller/timer circuit 43. The RV-EVENT, LV-EVENT, and RA-EVENT, LA-SENSE signals may be refractory or non-refractory and can inadvertently be triggered by electrical noise signals or aberrantly conducted depolarization waves rather than true R-waves or P-waves.

The techniques described in this disclosure, including those attributed to the IMD 16, the local computing device 140, and/or various constituent components, may be implemented, at least in part, in hardware, software, firmware, or any combination thereof. For example, various aspects of the techniques may be implemented within one or more processors, including one or more microprocessors, DSPs, ASICs, FPGAs, or any other equivalent integrated or discrete logic circuitry, as well as any combinations of such components, embodied in programmers, such as physician or patient programmers, stimulators, image processing devices, or other devices. The term “module,” “processor,” or “processing circuitry” may generally refer to any of the foregoing logic circuitry, alone or in combination with other logic circuitry, or any other equivalent circuitry.

Such hardware, software, and/or firmware may be implemented within the same device or within separate devices to support the various operations and functions described in this disclosure. In addition, any of the described units, modules, or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components or integrated within common or separate hardware or software components.

When implemented in software, the functionality ascribed to the systems, devices and techniques described in this disclosure may be embodied as instructions on a computer-readable medium such as RAM, ROM, NVRAM, EEPROM, FLASH memory, magnetic data storage media, optical data storage media, or the like. The instructions may be executed by processing circuitry and/or one or more processors to support one or more aspects of the functionality described in this disclosure.

### ILLUSTRATIVE EMBODIMENTS

Embodiment 1: A system comprising:
a computing apparatus comprising processing circuitry and configured to: obtain external electrical activity measured from tissue of a patient,
generate electrical heterogeneity information (EHI) based on the obtained electrical activity,
select a left ventricular pacing vector of a plurality of different left ventricular pacing vectors based on the generated EHI from the electrical activity monitored during delivery of pacing therapy using the plurality of different left ventricular pacing vectors, wherein each different left ventricular pacing vector uses a combination of pacing electrodes different from the remaining different left ventricular pacing vectors,
select one of adaptive pacing therapy or non-adaptive pacing therapy based on the generated EHI from the electrical activity monitored during delivery of adaptive pacing therapy using the selected left ventricular pacing vector, wherein the adaptive pacing therapy adjusts one or more pacing timing intervals based on one or both of intrinsic atrioventricular conduction and heart rate,
in response to selection of non-adaptive pacing therapy, select one interventricular pacing delay of a plurality of different interventricular pacing delays based on the generated EHI from the electrical activity monitored during delivery of pacing therapy using the plurality of different interventricular pacing delays using the selected left ventricular pacing vector,
in response to selection of adaptive pacing therapy, provide an indication that the pacing therapy is complete,
determine whether the generated EHI from the electrical activity monitored during the pacing therapy using the selected left ventricular pacing vector and the selected interventricular pacing delay indicates acceptable therapy,
in response to determination that the generated EHI from the electrical activity measured during the pacing therapy using the selected left ventricular pacing vector and the selected interventricular pacing delay does indicate acceptable therapy, provide an indication that the pacing therapy is complete, and
in response to determination that the generated EHI from the electrical activity monitored during the pacing therapy using the selected left ventricular pacing vector and the selected interventricular pacing delay does not indicate acceptable therapy, select one atrioventricular pacing delay of a plurality of different atrioventricular pacing delays based on the generated EHI from the electrical activity monitored during delivery of pacing therapy using the plurality of different atrioventricular pacing delays using the selected left ventricular pacing vector and the selected interventricular pacing delay.

Embodiment 2: A method for use in cardiac therapy evaluation comprising:
obtaining external electrical activity measured from tissue of a patient;
generating electrical heterogeneity information (EHI) based on the obtained electrical activity;
selecting a left ventricular pacing vector of a plurality of different left ventricular pacing vectors based on the generated EHI from the electrical activity measured during delivery of pacing therapy using the plurality of different left ventricular pacing vectors, wherein each different left ventricular pacing vector uses a combination of pacing electrodes different from the remaining different left ventricular pacing vectors;
selecting one of adaptive pacing therapy or non-adaptive pacing therapy based on the generated EHI from the electrical activity measured during delivery of adaptive pacing therapy using the selected left ventricular pacing vector, wherein the adaptive pacing therapy adjusts one or more pacing timing intervals based on one or both of intrinsic atrioventricular conduction and heart rate;
in response to selection of adaptive pacing therapy, providing an indication that the pacing therapy is complete;
in response to selection of non-adaptive pacing therapy, selecting one interventricular pacing delay of a plurality of different interventricular pacing delays based on the generated EHI from the electrical activity measured during delivery of pacing therapy using the plurality of different interventricular pacing delays using the selected left ventricular pacing vector;
determining whether the generated EHI from the electrical activity measured during the pacing therapy using the selected left ventricular pacing vector and the selected interventricular pacing delay indicates acceptable therapy;
in response to determination that the generated EHI from the electrical activity measured during the pacing therapy using the selected left ventricular pacing vector and the selected interventricular pacing delay does indicate acceptable therapy, providing an indication that the pacing therapy is complete; and
in response to determination that the generated EHI from the electrical activity measured during the pacing therapy using the selected left ventricular pacing vector and the selected interventricular pacing delay does not indicate acceptable therapy, selecting one atrioventricular pacing delay of a plurality of different atrioventricular pacing delays based on the generated EHI from the electrical activity measured during delivery of pacing therapy using the plurality of different atrioventricular pacing delays using the selected left ventricular pacing vector and the selected interventricular pacing delay.

Embodiment 3: The system as in embodiment 1 or the method as in embodiment 2, wherein the EHI comprises a standard deviation of electrical activation times (SDAT).

Embodiment 4: The system or method as in embodiment 3, wherein selecting a left ventricular pacing vector of a plurality of different left ventricular pacing vectors based on the generated EHI from the electrical activity measured during delivery of pacing therapy using the plurality of different left ventricular pacing vectors comprises selecting the left ventricular pacing vector of the plurality of different left ventricular pacing vectors that has the lowest SDAT.

Embodiment 5: The system or method as in any one of embodiments 1-4, wherein selecting one of adaptive pacing therapy or non-adaptive pacing therapy based on the generated EHI from the electrical activity measured during delivery of adaptive pacing therapy using the selected left ventricular pacing vector comprises:
determining whether the generated EHI from the electrical activity measured during delivery of adaptive pacing therapy using the selected left ventricular pacing vector indicates acceptable therapy;
selecting adaptive pacing therapy in response to the determination that the generated EHI indicates acceptable therapy; and
selecting non-adaptive pacing therapy in response to the determination that the generated EHI does not indicate acceptable therapy.

Embodiment 6: The system or method as in embodiment 5, wherein the EHI comprises a standard deviation of electrical activation times (SDAT) measured by the plurality of external electrodes,

wherein determining whether the generated EHI from the electrical activity measured during delivery of adaptive pacing therapy using the selected left ventricular pacing vector indicates acceptable therapy comprises determining whether the SDAT from the electrical activity measured during delivery of adaptive pacing therapy using the selected left ventricular pacing vector is less than or equal to an acceptable therapy threshold value.

Embodiment 7: The system or method as in embodiments 3-4 and 6, wherein selecting one interventricular pacing delay of a plurality of different interventricular pacing delays based on the generated EHI from the electrical activity measured during delivery of pacing therapy using the plurality of different interventricular pacing delays using the selected left ventricular pacing vector comprises selecting the interventricular pacing delay that has the lowest SDAT.

Embodiment 8: The system or method as in embodiments 3-4 and 6-7, wherein selecting one atrioventricular pacing delay of a plurality of different atrioventricular pacing delays based on the generated EHI from the electrical activity measured during delivery of pacing therapy using the plurality of different atrioventricular pacing delays using the selected left ventricular pacing vector and selected interventricular pacing delay comprises selecting the atrioventricular pacing delay that has the lowest SDAT.

Embodiment 9: The system or method as in any one of embodiments 1-8, wherein the plurality of external electrodes comprises surface electrodes positioned in an array configured to be located proximate the skin of the torso of the patient.

Embodiment 10: The system as in any one of embodiments 1 and 3-9, wherein the system further comprises display apparatus, wherein the display apparatus comprises a graphical user interface configured to present information for use in assisting a user in evaluating and adjusting cardiac therapy delivered to a patient,

wherein the computing apparatus is further configured to display one or more of the selected left ventricular pacing vector, the selected interventricular pacing delay, and the selected atrioventricular pacing delay on the graphical user interface.

Embodiment 11: The system as in embodiment 10, wherein the computing apparatus is further configured to display a graphical representation of surrogate electrical activation times measured by the plurality of external electrodes for at least one of the plurality of left ventricular pacing vectors.

Embodiment 12: The system or method as in any one of embodiments 1-9, wherein the pacing therapy is delivered by at least one implantable electrode coupled to at least one of a lead and a leadless implantable medical device.

Embodiment 13: A system comprising:
a computing apparatus comprising processing circuitry and configured to:
obtain external electrical activity measured from tissue of a patient,
generate electrical heterogeneity information (EHI) based on the obtained electrical activity, and
based on the generated EHI from the electrical activity measured during delivery of pacing therapy, determine a left ventricular pacing vector of a plurality of different left ventricular pacing vectors and select one of adaptive pacing therapy or non-adaptive pacing therapy, wherein the adaptive pacing therapy adjusts one or more pacing timing intervals based on one or both of intrinsic atrioventricular conduction and heart rate.

Embodiment 14: The system as in embodiment 13, wherein the computing apparatus is further configured to in response to selection of non-adaptive pacing therapy, determine one or both of an interventricular pacing delay and an atrioventricular pacing delay based on the generated EHI from the electrical activity measured during delivery of pacing therapy.

Embodiment 15: The system as in any one of embodiments 1 and 3-14, wherein the system further comprises electrode apparatus comprising a plurality of external electrodes to be located proximate the skin of the torso of the patient to measure the external electrical activity measured from tissue of the patient.

Embodiment 16: The method as in any one of embodiments 2 and 3-12, wherein obtaining external electrical activity from tissue of a patient comprising obtaining external electrical activity from tissue of the patient using a plurality of external electrodes to be located proximate the skin of the torso of the patient.

This disclosure has been provided with reference to illustrative embodiments and is not meant to be construed in a limiting sense. As described previously, one skilled in the art will recognize that other various illustrative applications may use the techniques as described herein to take advantage of the beneficial characteristics of the apparatus and methods described herein. Various modifications of the illustrative embodiments, as well as additional embodiments of the disclosure, will be apparent upon reference to this description.

1. A medical device system comprising:
a computing apparatus comprising processing circuitry and configured to:
obtain external electrical activity measured from tissue of a patient,
generate electrical heterogeneity information (EHI) based on the obtained electrical activity,
select a left ventricular pacing vector of a plurality of different left ventricular pacing vectors based on the generated EHI from the electrical activity measured during delivery of pacing therapy using the plurality of different left ventricular pacing vectors, wherein each different left ventricular pacing vector uses a combination of pacing electrodes different from the remaining different left ventricular pacing vectors,
select one of adaptive pacing therapy or non-adaptive pacing therapy based on the generated EHI from the electrical activity measured during delivery of adaptive pacing therapy using the selected left ventricular pacing vector, wherein the adaptive pacing therapy adjusts one or more pacing timing intervals based on one or both of intrinsic atrioventricular conduction and heart rate,
in response to selection of adaptive pacing therapy, provide an indication that the pacing therapy is complete,
in response to selection of non-adaptive pacing therapy, select one interventricular pacing delay of a plurality of different interventricular pacing delays based on the generated EHI from the electrical activity measured during delivery of pacing therapy using the plurality of different interventricular pacing delays using the selected left ventricular pacing vector,
determine whether the generated EHI from the electrical activity measured during the pacing therapy using the selected left ventricular pacing vector and the selected interventricular pacing delay indicates acceptable therapy,
in response to determination that the generated EHI from the electrical activity measured during the pacing therapy using the selected left ventricular pacing vector and the selected interventricular pacing delay does indicate acceptable therapy, provide an indication that the pacing therapy is complete, and
in response to determination that the generated EHI from the electrical activity measured during the pacing therapy using the selected left ventricular pacing vector and the selected interventricular pacing delay does not indicate acceptable therapy, select one atrioventricular pacing delay of a plurality of different atrioventricular pacing delays based on the generated EHI from the electrical activity measured during delivery of pacing therapy using the plurality of different atrioventricular pacing delays using the selected left ventricular pacing vector and the selected interventricular pacing delay.

2. The system as in claim 1, the system further comprising electrode apparatus comprising a plurality of external electrodes to be located proximate the skin of the torso of the patient to measure the external electrical activity measured from tissue of the patient.

3. The system as in claim 1, wherein the EHI comprises a standard deviation of electrical activation times (SDAT), and wherein selecting a left ventricular pacing vector of a plurality of different left ventricular pacing vectors based on the generated EHI from the electrical activity measured during delivery of pacing therapy using the plurality of different left ventricular pacing vectors comprises selecting the left ventricular pacing vector of the plurality of different left ventricular pacing vectors that has the lowest SDAT.

4. The system as in claim 1, wherein selecting one of adaptive pacing therapy or non-adaptive pacing therapy based on the generated EHI from the electrical activity measured during delivery of adaptive pacing therapy using the selected left ventricular pacing vector comprises:
determining whether the generated EHI from the electrical activity measured during delivery of adaptive pacing therapy using the selected left ventricular pacing vector indicates acceptable therapy;
selecting adaptive pacing therapy in response to the determination that the generated EHI indicates acceptable therapy; and
selecting non-adaptive pacing therapy in response to the determination that the generated EHI does not indicate acceptable therapy.

5. The system as in claim 4, wherein the EHI comprises a standard deviation of electrical activation times (SDAT),
wherein determining whether the generated EHI from the electrical activity measured during delivery of adaptive pacing therapy using the selected left ventricular pacing vector indicates acceptable therapy comprises determining whether the SDAT from the electrical activity measured during delivery of adaptive pacing therapy using the selected left ventricular pacing vector is less than or equal to an acceptable therapy threshold value.

6. The system as in claim 1, wherein the EHI comprises a standard deviation of electrical activation times (SDAT), and wherein selecting one interventricular pacing delay of a plurality of different interventricular pacing delays based on the generated EHI from the electrical activity measured during delivery of pacing therapy using the plurality of different interventricular pacing delays using the selected left ventricular pacing vector comprises selecting the interventricular pacing delay that has the lowest SDAT.

7. The system as in claim 1, wherein the EHI comprises a standard deviation of electrical activation times (SDAT), and wherein selecting one atrioventricular pacing delay of a plurality of different atrioventricular pacing delays based on the generated EHI from the electrical activity measured during delivery of pacing therapy using the plurality of different atrioventricular pacing delays using the selected left ventricular pacing vector and selected interventricular pacing delay comprises selecting the atrioventricular pacing delay that has the lowest SDAT.

8. The system as in claim 1, wherein the system further comprises display apparatus, wherein the display apparatus comprises a graphical user interface configured to present information for use in assisting a user in evaluating and adjusting cardiac therapy delivered to a patient,
wherein the computing apparatus is further configured to display one or more of the selected left ventricular pacing vector, the selected interventricular pacing delay, and the selected atrioventricular pacing delay on the graphical user interface.

9. The system as in claim 8, wherein the computing apparatus is further configured to display a graphical representation of surrogate electrical activation times measured by the plurality of external electrodes for at least one of the plurality of left ventricular pacing vectors on the graphical user interface.

10. A medical device system comprising:
a computing apparatus comprising processing circuitry and configured to:
obtain external electrical activity measured from tissue of a patient,
generate electrical heterogeneity information (EHI) based on the obtained electrical activity, and
based on the generated EHI from the electrical activity measured during delivery of pacing therapy, determine a left ventricular pacing vector of a plurality of different left ventricular pacing vectors and select one of adaptive pacing therapy or non-adaptive pacing therapy, wherein the adaptive pacing therapy adjusts one or more pacing timing intervals based on one or both of intrinsic atrioventricular conduction and heart rate.

11. The system as in claim 10, wherein the computing apparatus is further configured to:
in response to selection of non-adaptive pacing therapy, determine one or both of an interventricular pacing delay and an atrioventricular pacing delay based on the generated EHI from the electrical activity measured during delivery of pacing therapy.

12. A method comprising:
obtaining external electrical activity from tissue of a patient;
generating electrical heterogeneity information (EHI) based on the obtained electrical activity;
selecting a left ventricular pacing vector of a plurality of different left ventricular pacing vectors based on the generated EHI from the electrical activity measured during delivery of pacing therapy using the plurality of different left ventricular pacing vectors, wherein each different left ventricular pacing vector uses a combination of pacing electrodes different from the remaining different left ventricular pacing vectors;
selecting one of adaptive pacing therapy or non-adaptive pacing therapy based on the generated EHI from the electrical activity measured during delivery of adaptive pacing therapy using the selected left ventricular pacing vector, wherein the adaptive pacing therapy adjusts one or more pacing timing intervals based on one or both of intrinsic atrioventricular conduction and heart rate;
in response to selection of adaptive pacing therapy, providing an indication that the pacing therapy is complete;
in response to selection of non-adaptive pacing therapy, selecting one interventricular pacing delay of a plurality of different interventricular pacing delays based on the generated EHI from the electrical activity measured during delivery of pacing therapy using the plurality of different interventricular pacing delays using the selected left ventricular pacing vector;
determining whether the generated EHI from the electrical activity measured during the pacing therapy using the selected left ventricular pacing vector and the selected interventricular pacing delay indicates acceptable therapy;
in response to determination that the generated EHI from the electrical activity measured during the pacing therapy using the selected left ventricular pacing vector and the selected interventricular pacing delay does indicate acceptable therapy, providing an indication that the pacing therapy is complete; and
in response to determination that the generated EHI from the electrical activity measured during the pacing therapy using the selected left ventricular pacing vector and the selected interventricular pacing delay does not indicate acceptable therapy, selecting one atrioventricular pacing delay of a plurality of different atrioventricular pacing delays based on the generated EHI from the electrical activity measured during delivery of pacing therapy using the plurality of different atrioventricular pacing delays using the selected left ventricular pacing vector and the selected interventricular pacing delay.

13. The method as in claim 12, wherein obtaining external electrical activity from tissue of a patient comprising obtaining external electrical activity from tissue of the patient using a plurality of external electrodes to be located proximate the skin of the torso of the patient.

14. The method as in claim 12, wherein the EHI comprises a standard deviation of electrical activation times (SDAT), and wherein selecting a left ventricular pacing vector of a plurality of different left ventricular pacing vectors based on the generated EHI from the electrical activity measured during delivery of pacing therapy using the plurality of different left ventricular pacing vectors comprises selecting the left ventricular pacing vector of the plurality of different left ventricular pacing vectors that has the lowest SDAT.

15. The method as in claim 12, wherein selecting one of adaptive pacing therapy or non-adaptive pacing therapy based on the generated EHI from the electrical activity measured during delivery of adaptive pacing therapy using the selected left ventricular pacing vector comprises:
determining whether the generated EHI from the electrical activity measured during delivery of adaptive pacing therapy using the selected left ventricular pacing vector indicates acceptable therapy;
selecting adaptive pacing therapy in response to the determination that the generated EHI indicates acceptable therapy; and
selecting non-adaptive pacing therapy in response to the determination that the generated EHI does not indicate acceptable therapy.

16. The method as in claim 12, wherein the EHI comprises a standard deviation of electrical activation times (SDAT),
wherein determining whether the generated EHI from the electrical activity measured during delivery of adaptive pacing therapy using the selected left ventricular pacing vector indicates acceptable therapy comprises determining whether the SDAT from the electrical activity measured during delivery of adaptive pacing therapy using the selected left ventricular pacing vector is less than or equal to an acceptable therapy threshold value.

17. The method as in claim 12, wherein the EHI comprises a standard deviation of electrical activation times (SDAT), and wherein selecting one interventricular pacing delay of a plurality of different interventricular pacing delays based on the generated EHI from the electrical activity measured during delivery of pacing therapy using the plurality of different interventricular pacing delays using the selected left ventricular pacing vector comprises selecting the interventricular pacing delay that has the lowest SDAT.

18. The method as in claim 12, wherein the EHI comprises a standard deviation of electrical activation times (SDAT), and wherein selecting one atrioventricular pacing delay of a plurality of different atrioventricular pacing delays based on the generated EHI from the electrical activity measured during delivery of pacing therapy using the plurality of different atrioventricular pacing delays using the selected left ventricular pacing vector and selected interventricular pacing delay comprises selecting the atrioventricular pacing delay that has the lowest SDAT.

19. The method as claim 12, the method further comprising:
providing graphical user interface configured to present information for use in assisting a user in evaluating and adjusting cardiac therapy delivered to a patient; and
displaying one or more of the selected left ventricular pacing vector, the selected interventricular pacing delay, and the selected atrioventricular pacing delay on the graphical user interface.

20. The method as claim 12, the method further comprising displaying a graphical representation of surrogate electrical activation times measured by the plurality of external electrodes for at least one of the plurality of left ventricular pacing vectors on the graphical user interface.
